(19) United States
(12) Patent Application Publication
(10) Pub. No.: US 2009/0137681 A1
(43) Pub. Date: May 28, 2009

(54) Sirtuin Inhibiting Compounds

(76) Inventors: David A Sinclair, West Roxbury, MA (US); Antonello Mai, Rome (IT)

Correspondence Address: WOLF GREENFIELD & SACKS, P.C., 600 ATLANTIC AVENUE, BOSTON, MA, 02210-2206, US

(21) Appl. No.: 11/910,681

(22) PCT Filed: Apr. 6, 2006
(86) PCT No.: PCT/US2006/012795

§ 371 Date: Oct. 30, 2008

Related U.S. Application Data
(60) Provisional application No. 60/669,484, filed on Apr. 8, 2005.
(60) Provisional application No. 60/700,869, filed on Jul. 20, 2005.

(51) Int. Cl. A61K 31/18; C07C 237/30; C07C 251/00; C07C 311/00; A61K 31/165; A61K 31/13

(52) U.S. Cl. 514/603; 564/164; 564/270; 514/620; 514/641; 564/86

(57) ABSTRACT

Provided herein are compositions and methods for treating or preventing cancer and autoimmune diseases. Compositions comprise a sirtuin inhibitory compound that decreases the activity of a sirtuin, such as SIRT1 or Sir2. Exemplary methods comprise contacting a cell or a molecule with a sirtuin inhibitory compound that decreases the activity of a sirtuin and thereby reduces the life span of a cell, kills the cell or renders it susceptible to certain cell stresses including radiation and chemotherapy. Other methods include treating pathogens expressing a sirtuin.

### CROSS-REFERENCE TO RELATED APPLICATIONS

This application claims the benefit of U.S. Provisional Application No. 60/669,484, filed Apr. 8, 2005, and U.S. Provisional Application No. 60/700,869, filed Jul. 20, 2005, the content of each of which is specifically incorporated by reference herein in its entirety.

### GOVERNMENTAL SUPPORT

This invention was made with government support under Grant numbers RO1 GM068072 and RO0 AG19972 awarded by the National Institutes of Health. The government has certain rights in this invention.

### BACKGROUND

The reversible acetylation of histones is one of the best studied modes of gene regulation.¹⁻⁷ Specific patterns of gene expression are the result of the balance between the activities of histone acetyltransferases (HAT) and histone deacetylases (HDAC). The importance of this balance is underscored by the fact that perturbation of histone acetylation has been linked to several diseases including cancer.^{3,8,9} The majority of the genome in eukaryotes is maintained in a transcriptionally inactive or “silent” state and is associated with hypoacetylation of the ε-amino group of specific lysines in the histone tail, whereas transcriptionally active regions of the genome are typically hyperacetylated.

Three general classes of deacetylases have been identified in eukaryotes: class I, class II, and class III HDACs, also known as the sirtuins.^{10,11} Another deacetylase, maize HD2,¹² is structurally quite different from mammalian HDACs and has been attributed to a class of its own. Class I HDACs are predominantly nuclear and are expressed in most tissues and cell types. Class II HDACs are regulated by compartmentalization between the nucleus and cytoplasm through reversible phosphorylation, show a tissue-specific expression, and are subdivided into two subclasses, IIa and IIb, based on their sequence homology and domain organization. Class I and class II HDACs are targeted to specific chromatin domains, have a Zn⁺²-dependent mechanism of deacetylation to generate free acetate and the deacetylated protein.

Class I and II HDACs are inhibited by trichostatin A (TSA) and other natural and synthetic compounds,¹³ the majority of which are not able to distinguish between class I and class II HDACs. Exceptions include trapoxin (TPX)-A and -B,¹³ cyclic hydroxamic acid-containing peptide I (CHAP1),¹⁴ and sodium butyrate (NaB),¹⁵ which selectively inhibit class I and IIa HDACs. FK-228 specifically inhibits class I HDACs,¹⁶ and tubacin has been identified as a cell-selective HDAC6 inhibitor.¹⁷⁻²⁰ We recently described a series of aryloxopropenyl-pyrrolyl-hydroxyamides²¹ that selectively inhibit maize HD1-A, a class IIa HDAC.

Sirtuins are structurally and mechanistically distinct from other HDACs. They contain a conserved ˜300 amino acid catalytic domain which catalyzes a two step, NAD⁺-dependent deacetylation reaction. The first step is cleavage of the nicotinamide-ribose glycosidic bond of NAD⁺ with release of free nicotinamide and the formation of a relatively long-lived peptidyl-imidate intermediate.²² Second is the transfer of the acetyl group to ADP-ribose with production of O-acetyl-ADP-ribose.²³⁻²⁶

Yeast Sir2 (silent information regulator 2) is the founding member of the sirtuin family. Sir2 is required for transcriptional silencing at telomeres, ribosomal DNA, and the silent mating type loci.^{27,28} Sir2 and its homologues have recently gained attention for their ability to mimic the diet known as caloric restriction, which extends lifespan in a variety of organisms, including yeast,²⁹ Caenorhabditis elegans,³⁰ rodents,³¹ and probably primates.³² Sir2-like proteins can also deacetylate non-histone proteins, including the tumor suppressor p53,^{33,34} the RNA polymerase I transcription factor TAFI68,³⁵ the bovine serum albumin (BSA),³³ the microtubule protein α-tubulin in eukaryotes,³⁶ the archaeal chromatin protein Alba,³⁷ and the acetyl-coenzyme A synthetase (ACS) in prokaryotes.³⁸ In particular, the recently described role for Sir2 in the control of ACS establishes a link between transcription and intermediary metabolism.³⁹

There is considerable interest in understanding the possible function of the novel product of the Sir2 reaction, O-acetyl-ADP-ribose (OAADPr). 2′-OAADPr is the initial enzymatic product which then equilibrates with 3′-OAADPr in solution through a nonenzymatic, intramolecular transesterification reaction.²²⁻²⁵ The physiological role of 2′- and 3′-OAADPr is still unclear, but microinjection of these compounds has been reported to delay or block the oocyte maturation and the cell cycle in embryonic development.⁴⁰

Class I/II HDAC inhibitors are ineffective in inhibiting sirtuins, nevertheless they have been shown to induce differential changes in gene expression of SIRT mRNAs in cultured neuronal cells, with up-regulation of SIRT2, -4, and -7 and down-regulation of SIRT1, -5, and -6 mRNAs.⁴¹ A few specific sirtuin inhibitors have been reported to date, including sirtinol 1,⁴² splitomicin,⁴³ and nicotinamide⁴⁴.

A number of splitomicin derivatives and analogues have been prepared and evaluated as Sir2 inhibitors,^{45,46} and a variety of 3-substituted pyridines have been found to be substrate for the Sir2-catalyzed transglycosidation,⁴⁷ whereas little attention has been devoted to sirtinol 1 and its analogues. Nevertheless, the 1 structure, having a 2-hydroxy-1-naphtaldehyde moiety linked to a 2-amino-N-(1-phenylethyl)benzamide portion through a aldimine linkage, can be a interesting chemical template to develop new 1 analogues. Such compounds could provide insight to the inhibitory mechanism of 1, and may be useful tools for functional characterization and/or elucidation of the in vivo functions of these enzymes.

### SUMMARY

Provided herein are sirtuin inhibitory compounds, such as compounds having any one of formulas I-XI, a salt, or a prodrug thereof. The sirtuin-inhibitory compound preferably decreases human Sir2, e.g., SIRT1 and SIRT2, protein activity. Pharmaceutical compositions comprising a sirtuin inhibitory compound and a pharmaceutically acceptable vehicle are also provided herein.

Also provided are compositions comprising a first agent that is a sirtuin inhibitory compound having any one of formulas I-XI and a second agent. The second agent may be a compound having any one of formulas I-XI that is different from the first agent. The second agent may be an agent that induces cell death, such as a chemotherapeutic agent.

Further provided herein are methods for inhibiting a sirtuin. A method may comprise contacting a sirtuin with a sirtuin inhibitory compound. The sirtuin may be SIRT1. A sirtuin may be in a cell and the method may comprise contacting the cell with a sirtuin inhibitory compound. A cell may be in vitro, ex vivo or in vivo.

Also provided are methods for reducing the lifespan of a cell, for killing a cell, or for rendering a cell more sensitive to stress. A method may comprise contacting the cell with a sirtuin inhibitory compound. The stress may be exposure to an agent that induces cell death.

When the cell is in vivo, the method may be used for treating or preventing a condition associated with the presence of undesirable cells in a subject. A method may comprise administering to a subject in need thereof a therapeutically effective amount of a sirtuin inhibitory compound. The condition may be cancer or an autoimmune disease. A method may further comprise administering to the subject another agent, such as a chemotherapeutic agent.

### BRIEF DESCRIPTION OF FIGURES

FIG. 1 shows the structures of sirtinol analogues described herein.

FIG. 2 is a schematic representation of the yeast Sir2-regulated telomeric silencing assay. Degree of Sir2 activity in vivo may be determined using a strain with URA3 integrated at a Sir2-silenced telomeric locus. Lack of growth on 5-FOA medium is indicative of decreased Sir2 activity and a concomitant increase in URA3 expression.

FIG. 3 depicts the important interactions observed in the Sir2-Af2/NAD⁺/p53 ternary complex.

FIG. 4 depicts the role of the His118 and Ac—K→NAD⁺ acetyl transfer mechanism.

### DETAILED DESCRIPTION

### Definitions

A “form that is naturally occurring” when referring to a compound means a compound that is in a form, e.g., a composition, in which it can be found naturally. A compound is not in a form that is naturally occurring if, e.g., the compound has been purified and separated from at least some of the other molecules that are found with the compound in nature.

“Inhibiting a sirtuin protein” refers to the action of reducing at least one of the biological activities of a sirtuin protein to at least some extent, e.g., at least about 10%, 50%, 2 fold, 5 fold, 10 fold, 30 fold, 100 fold or more.

A “naturally occurring compound” refers to a compound that can be found in nature, i.e., a compound that has not been designed by man. A naturally occurring compound may have been made by man or by nature. A “non-naturally occurring compound” is a compound that is not known to exist in nature or that does not occur in nature.

“Sirtuin deacetylase protein family members;” “Sir2 family members;” “Sir2 protein family members;” or “sirtuin proteins” includes yeast Sir2, Sir-2.1, and human SIRT1 and SIRT2 proteins. The nucleotide and amino acid sequences of the human sirtuin, SIRT1 (silent mating type information regulation 2 homolog), are set forth as SEQ ID NOs: 1 and 2, respectively (corresponding to GenBank Accession numbers NM_{—}012238 and NP_{—}036370, respectively). The mouse homolog of SIRT1 is Sirt2α. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has.

SEQ ID NOs of the human genes referred to herein are identified in the table below:

| | | nucleotide sequence | | amino acid sequence | |
|---|---|---|---|---|---|
| | | | SEQ | | SEQ |
| name | | GenBank | ID NO | GenBank | ID NO |
| SIRT1 | | NM_012238 | 1 | NP_036370 | 2 |
| SIRT2 | i1 | NM_012237 | 3 | NP_036369 | 4 |
| | i2 | NM_030593 | 5 | NP_085096 | 6 |
| SIRT3 | ia | NM_012239 | | NP_036371 | |
| | ib | NM_001017524 | | NP_001017524 | |
| SIRT4 | | NM_012240 | | NP_036372 | |
| SIRT5 | i1 | NM_012241 | | NP_036373 | |
| | i2 | NM_031244 | | NP_112534 | |
| SIRT6 | | NM_016539 | | NP_057623 | |
| SIRT7 | | NM_016538 | | NP_057622 | |

“Biologically active portion of a sirtuin” refers to a portion of a sirtuin protein having a biological activity, such as the ability to deacetylate. Biologically active portions of sirtuins may comprise the core domain of sirtuins. For example, amino acids 62-293 of SIRT1 having SEQ ID NO: 2, which are encoded by nucleotides 237 to 932 of SEQ ID NO: 1, encompass the NAD⁺ binding domain and the substrate binding domain. Therefore, this region is sometimes referred to as the core domain. Other biologically active portions of SIRT1, also sometimes referred to as core domains, include about amino acids 261 to 447 of SEQ ID NO: 2, which are encoded by nucleotides 834 to 1394 of SEQ ID NO: 1; about amino acids 242 to 493 of SEQ ID NO: 2, which are encoded by nucleotides 777 to 1532 of SEQ ID NO: 1; or about amino acids 254 to 495 of SEQ ID NO: 2, which are encoded by nucleotides 813 to 1538 of SEQ ID NO: 1.

“Stress” refers to any non-optimal condition for growth, development or reproduction. A “stress condition” can be exposure to heatshock; osmotic stress; a DNA damaging agent; inadequate salt level; inadequate nitrogen levels; inadequate nutrient level; radiation or a toxic compound, e.g., a toxin or chemical warfare agent (such as dirty bombs and other weapons that may be used in bioterrorism). “Inadequate levels” refer to levels that result in non-optimal condition for growth, development or reproduction.

The term “stereoisomers” is art-recognized and refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

Furthermore, a “stereoselective process” is one which produces a particular stereoisomer of a reaction product in preference to other possible stereoisomers of that product. An “enantioselective process” is one which favors production of one of the two possible enantiomers of a reaction product.

The term “regioisomers” is art-recognized and refers to compounds which have the same molecular formula but differ in the connectivity of the atoms. Accordingly, a “regioselective process” is one which favors the production of a particular regioisomer over others, e.g., the reaction produces a statistically significant increase in the yield of a certain regioisomer.

The term “epimers” is art-recognized and refers to molecules with identical chemical constitution and containing more than one stereocenter, but which differ in configuration at only one of these stereocenters.

“Treating” a condition or disease refers to curing as well as ameliorating at least one symptom of the condition or disease or preventing the condition or disease from worsening.

The term “structure-activity relationship” or “(SAR)” is art-recognized and refers to the way in which altering the molecular structure of a drug or other compound alters its biological activity, e.g., its interaction with a receptor, enzyme, nucleic acid or other target and the like.

The term “aliphatic” is art-recognized and refers to a linear, branched, cyclic alkane, alkene, or alkyne. In certain embodiments, aliphatic groups in the present compounds are linear or branched and have from 1 to about 20 carbon atoms.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas:

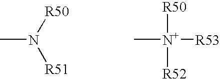

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

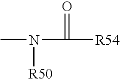

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

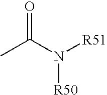

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas:

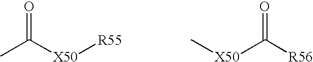

wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “carbamoyl” refers to —O(C═O)NRR′, where R and R′ are independently H, aliphatic groups, aryl groups or heteroaryl groups.

The term “oxo” refers to a carbonyl oxygen (═O).

The terms “oxime” and “oxime ether” are art-recognized and refer to moieties that may be represented by the general formula:

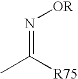

wherein R75 is hydrogen, alkyl, cycloalkyl, alkenyl, alkynyl, aryl, aralkyl, or —(CH₂)ₘ—R61. The moiety is an “oxime” when R is H; and it is an “oxime ether” when R is alkyl, cycloalkyl, alkenyl, alkynyl, aryl, aralkyl, or —(CH₂)ₘ—R61.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula:

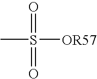

in which R57 is an electron pair, hydrogen, alkyl, cycloalkyl, or aryl.

The term “sulfate” is art recognized and includes a moiety that may be represented by the general formula:

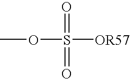

in which R57 is as defined above.

The term “sulfonamido” is art recognized and includes a moiety that may be represented by the general formula:

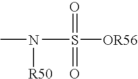

in which R50 and R56 are as defined above.

The term “sulfamoyl” is art-recognized and refers to a moiety that may be represented by the general formula:

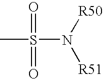

in which R50 and R51 are as defined above.

The term “sulfonyl” is art-recognized and refers to a moiety that may be represented by the general formula:

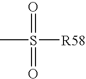

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

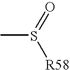

in which R58 is defined above.

The term “phosphoryl” is art-recognized and may in general be represented by the formula:

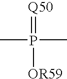

wherein Q50 represents S or O, and R59 represents hydrogen, a lower alkyl or an aryl. When used to substitute, e.g., an alkyl, the phosphoryl group of the phosphorylalkyl may be represented by the general formulas:

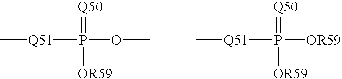

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

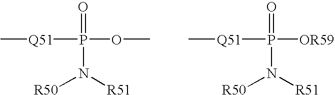

wherein Q51, R50, R51 and R59 are as defined above.

The term “phosphonamidite” is art-recognized and may be represented in the general formulas:

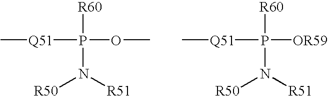

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 3^{rd} ed.; Wiley: New York, 1999). Protected forms of the inventive compounds are included within the scope of this invention.

For purposes of this invention, the chemical elements are identified in accordance with the Periodic Table of the Elements, CAS version, Handbook of Chemistry and Physics, 67th Ed., 1986-87, inside cover.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2^{nd} ed., Wiley: New York, 1991).

The term “hydroxyl-protecting group” is art-recognized and refers to those groups intended to protect a hydroxyl group against undesirable reactions during synthetic procedures and includes, for example, benzyl or other suitable esters or ethers groups known in the art.

The term “carboxyl-protecting group” is art-recognized and refers to those groups intended to protect a carboxylic acid group, such as the C-terminus of an amino acid or peptide or an acidic or hydroxyl azepine ring substituent, against undesirable reactions during synthetic procedures and includes. Examples for protecting groups for carboxyl groups involve, for example, benzyl ester, cyclohexyl ester, 4-nitrobenzyl ester, t-butyl ester, 4-pyridylmethyl ester, and the like.

The term “amino-blocking group” is art-recognized and refers to a group which will prevent an amino group from participating in a reaction carried out on some other functional group, but which can be removed from the amine when desired. Such groups are discussed by in Ch. 7 of Greene and Wuts, cited above, and by Barton, Protective Groups in Organic Chemistry ch. 2 (McOmie, ed., Plenum Press, New York, 1973). Examples of suitable groups include acyl protecting groups such as, to illustrate, formyl, dansyl, acetyl, benzoyl, trifluoroacetyl, succinyl, methoxysuccinyl, benzyl and substituted benzyl such as 3,4-dimethoxybenzyl, o-nitrobenzyl, and triphenylmethyl; those of the formula —COOR where R includes such groups as methyl, ethyl, propyl, isopropyl, 2,2,2-trichloroethyl, 1-methyl-1-phenylethyl, isobutyl, t-butyl, t-amyl, vinyl, allyl, phenyl, benzyl, p-nitrobenzyl, o-nitrobenzyl, and 2,4-dichlorobenzyl; acyl groups and substituted acyl such as formyl, acetyl, chloroacetyl, dichloroacetyl, trichloroacetyl, trifluoroacetyl, benzoyl, and p-methoxybenzoyl; and other groups such as methanesulfonyl, p-toluenesulfonyl, p-bromobenzenesulfonyl, p-nitrophenylethyl, and p-toluenesulfonyl-aminocarbonyl. Preferred amino-blocking groups are benzyl (—CH₂C₆H₅), acyl [C(O)R1] or SiR1₃ where R1 is C₁-C₄ alkyl, halomethyl, or 2-halo-substituted-(C₂-C₄ alkoxy), aromatic urethane protecting groups as, for example, carbonylbenzyloxy (Cbz); and aliphatic urethane protecting groups such as t-butyloxycarbonyl (Boc) or 9-fluorenylmethoxycarbonyl (FMOC).

The definition of each expression, e.g. lower alkyl, m, n, p and the like, when it occurs more than once in any structure, is intended to be independent of its definition elsewhere in the same structure.

The term “electron-withdrawing group” is art-recognized, and refers to the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59 (McGraw Hill Book Company: New York, 1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays described herein. The term “small organic molecule” refers to a small molecule that is often identified as being an organic or medicinal compound, and does not include molecules that are exclusively nucleic acids, peptides or polypeptides.

The term “chemical entity,” as used herein, refers to chemical compounds, complexes of two or more chemical compounds, and fragments of such compounds or complexes. In certain instances, it is desirable to use chemical entities exhibiting a wide range of structural and functional diversity, such as compounds exhibiting different shapes (e.g., flat aromatic rings(s), puckered aliphatic rings(s), straight and branched chain aliphatics with single, double, or triple bonds) and diverse functional groups (e.g., carboxylic acids, esters, ethers, amines, aldehydes, ketones, and various heterocyclic rings).

As used herein the term “docking” refers to a process of placing a chemical entity in close proximity with a druggable region, or a process of finding low energy conformations of a chemical entity/druggable region complex.

The term “druggable region”, when used in reference to a polypeptide, nucleic acid, complex and the like, refers to a region of the molecule which is a target or is a likely target for binding a modulator. For a polypeptide, a druggable region generally refers to a region wherein several amino acids of a polypeptide would be capable of interacting with a modulator or other molecule. For a polypeptide or complex thereof, exemplary druggable regions include binding pockets and sites, enzymatic active sites, interfaces between domains of a polypeptide or complex, surface grooves or contours or surfaces of a polypeptide or complex which are capable of participating in interactions with another molecule. In certain instances, the interacting molecule is another polypeptide, which may be naturally-occurring. In other instances, the druggable region is on the surface of the molecule.

Druggable regions may be described and characterized in a number of ways. For example, a druggable region may be characterized by some or all of the amino acids that make up the region, or the backbone atoms thereof, or the side chain atoms thereof (optionally with or without the Cα atoms). Alternatively, in certain instances, the volume of a druggable region corresponds to that of a carbon based molecule of at least about 200 amu and often up to about 800 amu. In other instances, it will be appreciated that the volume of such region may correspond to a molecule of at least about 600 amu and often up to about 1600 amu or more.

Alternatively, a druggable region may be characterized by comparison to other regions on the same or other molecules. For example, the term “affinity region” refers to a druggable region on a molecule (such as a polypeptide of the invention) that is present in several other molecules, in so much as the structures of the same affinity regions are sufficiently the same so that they are expected to bind the same or related structural analogs. An example of an affinity region is an ATP-binding site of a protein kinase that is found in several protein kinases (whether or not of the same origin). The term “selectivity region” refers to a druggable region of a molecule that may not be found on other molecules, in so much as the structures of different selectivity regions are sufficiently different so that they are not expected to bind the same or related structural analogs. An exemplary selectivity region is a catalytic domain of a protein kinase that exhibits specificity for one substrate. In certain instances, a single modulator may bind to the same affinity region across a number of proteins that have a substantially similar biological function, whereas the same modulator may bind to only one selectivity region of one of those proteins.

Continuing with examples of different druggable regions, the term “undesired region” refers to a druggable region of a molecule that upon interacting with another molecule results in an undesirable affect. For example, a binding site that oxidizes the interacting molecule (such as P-450 activity) and thereby results in increased toxicity for the oxidized molecule may be deemed a “undesired region”. Other examples of potential undesired regions includes regions that upon interaction with a drug decrease the membrane permeability of the drug, increase the excretion of the drug, or increase the blood brain transport of the drug. It may be the case that, in certain circumstances, an undesired region will no longer be deemed an undesired region because the affect of the region will be favorable, e.g., a drug intended to treat a brain condition would benefit from interacting with a region that resulted in increased blood brain transport, whereas the same region could be deemed undesirable for drugs that were not intended to be delivered to the brain.

When used in reference to a druggable region, the “selectivity” or “specificity’ of a molecule such as a modulator to a druggable region may be used to describe the binding between the molecule and a druggable region. For example, the selectivity of a modulator with respect to a druggable region may be expressed by comparison to another modulator, using the respective values of K_{d} (i.e., the dissociation constants for each modulator-druggable region complex) or, in cases where a biological effect is observed below the K_{d}, the ratio of the respective EC₅₀'s (i.e., the concentrations that produce 50% of the maximum response for the modulator interacting with each druggable region).

### Sirtuin Inhibitors

Provided herein are compounds that inhibit a sirtuin deacetylase protein family member (referred to herein as a “sirtuin protein”). Exemplary sirtuin deacetylase proteins include the yeast silent information regulator (Sir2) and human SIRT1. Exemplary sirtuin inhibitory compounds are set forth in formulas I-M.

A sirtuin inhibitory compound may have the formula I:

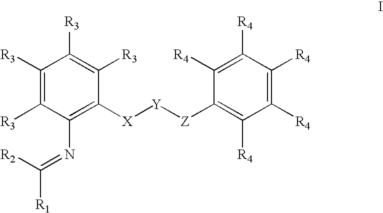

wherein, independently for each occurrence,

X is —O—, —N(Rₐ)—, —C(Rₐ)₂—, —C(═O)—, —C(═NR_{b})—, —C(═S)—, —S—, —S(═O)— or —S(═O)₂—;

Y is —O—, —N(Rₐ)—, C(Rₐ)₂—, —C(═O)—, —C(═NR_{b})—, —C(═S)—, —S—, —S(═O)— or —S(═O)₂—;

Z is —O—, —N(Rₐ)—, —C(Rₐ)₂—, —C(═O)—, —C(═NR_{b})—, —C(═S)—, —S—, —S(═O)— or —S(═O)₂—;

Rₐ is hydrogen, alkyl, aryl, or aralkyl;

R_{b} is hydrogen, hydroxyl, alkoxyl, amine, alkyl, aryl, or aralkyl;

R₁ is aryl;

R₂ is hydrogen, alkyl, aryl, or aralkyl;

R₃ is hydrogen, halogen, alkyl, alkenyl, alkynyl, aryl, heteroaryl, aralkyl, aralkyenyl, arallynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl, or sulfoxido;

R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl, or sulfoxido;

provided that when X is —C(═O)—; Y is —N(H)—; Z is —CH(CH₃)—; R₂ is hydrogen; R₃ is hydrogen; and R₄ is hydrogen; R₁ is not

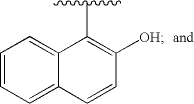

the compound is achiral or, when chiral, is a single stereoisomer or a mixture of stereoisomers.

In another aspect, a sirtuin inhibitory compound may be a compound of formula II:

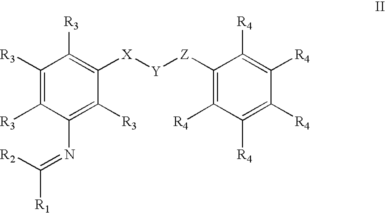

wherein, independently for each occurrence,

X is —O—, —N(Rₐ)—, —C(Rₐ)₂—, —C(═O)—, —C(═NR_{b})—, —C(═S)—, —S—, —S(═O)— or —S(═O)₂—;

Y is —O—, —N(Rₐ)—, —C(Rₐ)₂—, —C(═O)—, —C(═NR_{b})—, —C(═S)—, —S—, —S(═O)— or —S(═O)₂—;

Z is —O—, —N(Rₐ)—, —C(Rₐ)₂—, —C(═O)—, —C(═NR_{b})—, —C(═S)—, —S—, —S(═O)— or —S(═O)₂—;

Rₐ is hydrogen, alkyl, aryl, or aralkyl;

R_{b} is hydrogen, hydroxyl, alkoxyl, amine, alkyl, aryl, or aralkyl;

R₁ is aryl;

R₂ is hydrogen, alkyl, aryl, or aralkyl;

R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido; and

the compound is achiral or, when chiral, is a single stereoisomer or a mixture of stereoisomers.

In another aspect, a sirtuin inhibitory compound may be a compound of formula III:

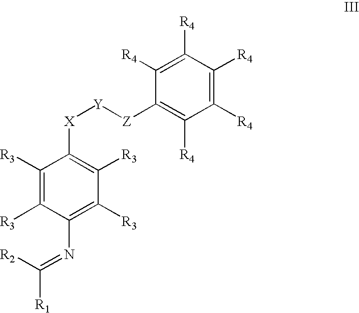

wherein, independently for each occurrence,

X is —O—, N(Rₐ)—, C(Rₐ)₂—, —C(═O)—, —C(═NR_{b})—, —C(═S)—, —S—, —S(═O)— or —S(═O)₂—;

Y is —O—, —N(Rₐ)—, —C(Rₐ)₂—, —C(═O)—, —C(═NR_{b})—, —C(═S)—, —S—, —S(═O)— or —S(═O)₂—;

Z is —O—, —N(Rₐ)—, —C(Rₐ)₂—, —C(═O)—, —C(═NR_{b})—, —C(═S)—, —S—, —S(═O)— or —S(═O)₂—;

Rₐ is hydrogen, alkyl, aryl, or aralkyl;

R_{b} is hydrogen, hydroxyl, alkoxyl, amine, alkyl, aryl, or aralkyl;

R₁ is aryl;

R₂ is hydrogen, alkyl, aryl, or aralkyl;

R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido; and

the compound is achiral or, when chiral, is a single stereoisomer or a mixture of stereoisomers.

In certain embodiments, sirtuin inhibitory compounds are represented by I, II, or III and the attendant definitions, wherein X is —C(═O)—, —N(H)—, —S— or —S(═O)₂—.

In certain embodiments, sirtuin inhibitory compounds are represented by I, II, or III and the attendant definitions, wherein Y is —C(═O)—, —N(H)— or —CH₂—.

In certain embodiments, sirtuin inhibitory compounds are represented by I, II, or III and the attendant definitions, wherein Z is —CH(CH₃)—.

In certain embodiments, sirtuin inhibitory compounds are represented by I, II, or III and the attendant definitions, wherein R₂ is hydrogen.

In certain embodiments, sirtuin inhibitory compounds are represented by I, II, or III and the attendant definitions, wherein R₃ is hydrogen.

In certain embodiments, sirtuin inhibitory compounds are represented by I, II, or III and the attendant definitions, wherein R₄ is hydrogen.

In certain embodiments, sirtuin inhibitory compounds are represented by I, II, or III and the attendant definitions, wherein R₂ is hydrogen; R₃ is hydrogen; and R₄ is hydrogen.

In another aspect, a sirtuin inhibitory compound may be a compound of formula IV:

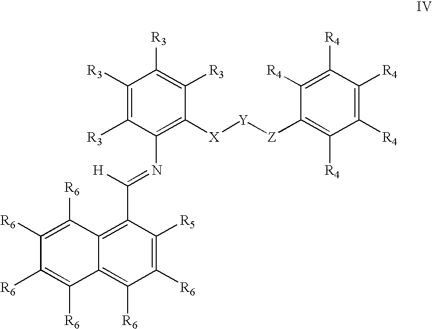

wherein, independently for each occurrence,

X is —O—, —N(Rₐ)—, —C(Rₐ)₂—, —C(═O)—, —C(═NR_{b})—, —C(═S)—, —S—, —S(═O)— or —S(═O)₂—;

Y is —O—, N(Rₐ)—, C(Rₐ)₂—, —C(═O)—, —C(═NR_{b})—, —C(═S)—, —S—, —S(═O)— or —S(═O)₂—;

Z is —O—, —N(Rₐ)—, —C(Rₐ)₂—, —C(═O)—, —C(═NR_{b})—, —C(═S)—, —S—, —S(═O)— or —S(═O)₂—;

Rₐ is hydrogen, alkyl, aryl, or aralkyl;

R_{b} is hydrogen, hydroxyl, alkoxyl, amine, alkyl, aryl, or aralkyl;

R₁ is aryl;

R₃ is hydrogen, halogen, allyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₅ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₆ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

provided that when X is —C(═O)—; Y is —N(H)—; Z is —CH(CH₃)—; R₃ is hydrogen; R₄ is hydrogen; and R₆ is hydrogen; R₅ is not hydroxyl; and

the compound is achiral or, when chiral, is a single stereoisomer or a mixture of stereoisomers.

In another aspect, a sirtuin inhibitory compound may be a compound of formula V:

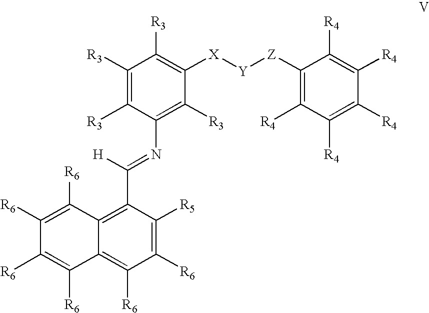

wherein, independently for each occurrence,

X is —O—, N(Rₐ)—, —C(Rₐ)₂—, —C(═O)—, —C(═NR_{b})—, —C(═S)—, —S—, —S(═O)— or —S(═O)₂—;

Y is —O—, N(Rₐ)—, —C(Rₐ)₂—, —C(═O)—, —C(═NR_{b})—, —C(═S)—, —S—, —S(═O)— or —S(═O)₂—;

Z is —O—, —N(Rₐ)—, —C(Rₐ)₂—, —C(═O)—, —C(═NR_{b})—, —C(═S)—, —S—, —S(═O)— or —S(═O)₂—;

Rₐ is hydrogen, alkyl, aryl, or aralkyl;

R_{b} is hydrogen, hydroxyl, alkoxyl, amine, alkyl, aryl, or aralkyl;

R₁ is aryl;

R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₅ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₆ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido; and

the compound is achiral or, when chiral, is a single stereoisomer or a mixture of stereoisomers.

In another aspect, a sirtuin inhibitory compound may be a compound of formula VI:

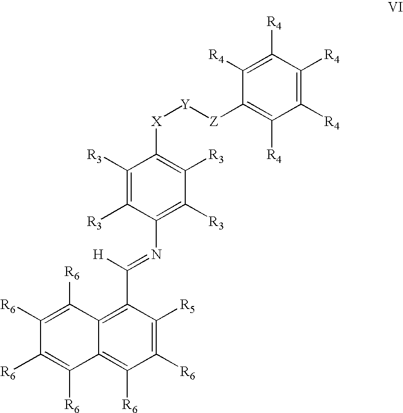

wherein, independently for each occurrence,

X is —O—, —N(Rₐ)—, —C(Rₐ)₂—, —C(═O)—, —C(═NR_{b})—, —C(═S)—, —S—, —S(═O)— or —S(═O)₂—;

Y is —O—, —N(Rₐ)—, —C(Rₐ)₂—, —C(═O)—, —C(═NR_{b})—, —C(═S)—, —S—, —S(═O)— or —S(═O)₂—;

Z is —O—, —N(Rₐ)—, —C(Rₐ)₂—, —C(═O)—, —C(═NR_{b})—, —C(═S)—, —S—, —S(═O)— or —S(═O)₂—;

Rₐ is hydrogen, alkyl, aryl, or aralkyl;

R_{b} is hydrogen, hydroxyl, alkoxyl, amine, alkyl, aryl, or aralkyl;

R₁ is aryl;

R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₅ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₆ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido; and

the compound is achiral or, when chiral, is a single stereoisomer or a mixture of stereoisomers.

In certain embodiments, sirtuin inhibitory compounds are represented by IV, V, or VI and the attendant definitions, wherein X is —C(═O)—, —N(Rₐ)—, —S— or —S(═O)₂—.

In certain embodiments, sirtuin inhibitory compounds are represented by IV, V, or VI and the attendant definitions, wherein X is —C(═O)—, —N(H)—, —S— or —S(═O)₂—.

In certain embodiments, sirtuin inhibitory compounds are represented by IV, V, or VI and the attendant definitions, wherein Y is —C(═O)—, —N(Rₐ)— or —C(Rₐ)₂—.

In certain embodiments, sirtuin inhibitory compounds are represented by IV, V, or VI and the attendant definitions, wherein Y is —C(═O)—, —N(H)— or —CH₂—.

In certain embodiments, sirtuin inhibitory compounds are represented by IV, V, or VI and the attendant definitions, wherein Z is —C(Rₐ)₂—.

In certain embodiments, sirtuin inhibitory compounds are represented by IV, V, or VI and the attendant definitions, wherein Z is —CH(Rₐ)—; and Rₐ is alkyl.

In certain embodiments, sirtuin inhibitory compounds are represented by IV, V, or VI and the attendant definitions, wherein Z is —CH(CH₃)—.

In certain embodiments, sirtuin inhibitory compounds are represented by IV, V, or VI and the attendant definitions, wherein R₃ is hydrogen.

In certain embodiments, sirtuin inhibitory compounds are represented by IV, V, or VI and the attendant definitions, wherein R₄ is hydrogen.

In certain embodiments, sirtuin inhibitory compounds are represented by IV, V, or VI and the attendant definitions, wherein R₅ is hydroxyl.

In certain embodiments, sirtuin inhibitory compounds are represented by IV, V, or VI and the attendant definitions, wherein R₆ is hydrogen.

In certain embodiments, sirtuin inhibitory compounds are represented by IV, V, or VI and the attendant definitions, wherein R₅ is hydroxyl; and R₆ is hydrogen.

In certain embodiments, sirtuin inhibitory compounds are represented by IV, V, or VI and the attendant definitions, wherein R₅ is hydroxyl; R₆ is hydrogen; and R₄ is hydrogen.

In certain embodiments, sirtuin inhibitory compounds are represented by IV, V, or VI and the attendant definitions, wherein R₅ is hydroxyl; R₆ is hydrogen; R₄ is hydrogen; and R₃ is hydrogen.

In certain embodiments, sirtuin inhibitory compounds are represented by IV, V, or VI and the attendant definitions, wherein R₅ is hydroxyl; R₆ is hydrogen; R₄ is hydrogen; R₃ is hydrogen; Z is —CH(Rₐ)—; and Rₐ is alkyl.

In certain embodiments, sirtuin inhibitory compounds are represented by IV, V, or VI and the attendant definitions, wherein R₅ is hydroxyl; R₆ is hydrogen; R₄ is hydrogen; R₃ is hydrogen; and Z is —CH(CH₃)—.

In another aspect, a sirtuin inhibitory compound may be a compound of formula VII:

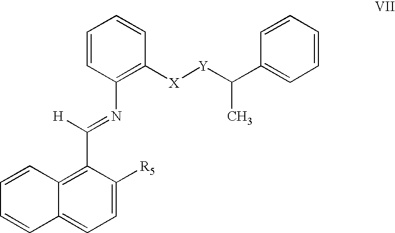

wherein, independently for each occurrence,

X is —N(H)—, —C(═O)—, —S— or —S(═O)₂—;

Y is —N(H)—, —CH₂— or —C(═O)—;

R₅ is hydrogen, hydroxyl or alkoxyl;

provided that when X is —C(═O)—; and Y is —N(H)—; R₅ is not hydroxyl; and

the compound is achiral or, when chiral, is a single stereoisomer or a mixture of stereoisomers.

In another aspect, a sirtuin inhibitory compound may be a compound of formula VIII:

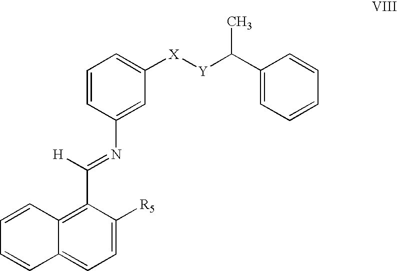

wherein, independently for each occurrence,

X is —N(H)—, —C(═O)—, —S— or —S(═O)₂—;

Y is —N(H)—, —CH₂— or —C(═O)—;

R₅ is hydrogen, hydroxyl or alkoxyl; and

the compound is achiral or, when chiral, is a single stereoisomer or a mixture of stereoisomers.

In another aspect, a sirtuin inhibitory compound may be a compound of formula IX:

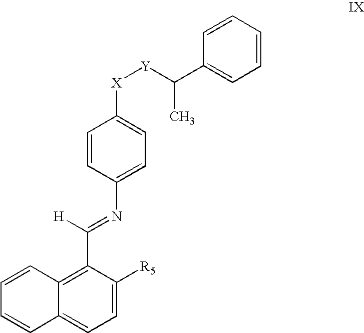

wherein, independently for each occurrence,

X is —N(H)—, —C(═O)—, —S— or —S(═O)₂—;

Y is —N(H)—, —CH₂— or —C(═O)—;

R₅ is hydrogen, hydroxyl or alkoxyl; and

the compound is achiral or, when chiral, is a single stereoisomer or a mixture of stereoisomers.

In certain embodiments, sirtuin inhibitory compounds are represented by VII, VIII, or IX and the attendant definitions, wherein R₅ is hydroxyl.

In certain embodiments, sirtuin inhibitory compounds are represented by VII, VIII, or IX and the attendant definitions, wherein R₅ is hydroxyl; X is —C(═O)—; and Y is —N(H)—.

In certain embodiments, sirtuin inhibitory compounds are represented by VII, VIII, or IX and the attendant definitions, wherein R₅ is hydroxyl; X is —N(H)—; and Y is —C(═O)—.

In certain embodiments, sirtuin inhibitory compounds are represented by VII, VIII, or IX and the attendant definitions, wherein R₅ is hydroxyl; X is —S—; and Y is —CH₂—.

In certain embodiments, sirtuin inhibitory compounds are represented by VII, VIII, or IX and the attendant definitions, wherein R₅ is hydroxyl; X is —S(═O)₂—; and Y is —N(H)—.

In certain embodiments, sirtuin inhibitory compounds are represented by VII, VIII, or IX and the attendant definitions, wherein the compound is a single enantiomer or stereoisomer.

In another aspect, a sirtuin inhibitory compound may be a compound of formula X:

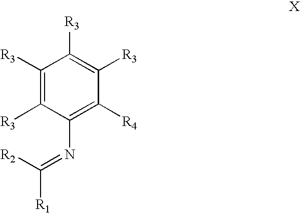

wherein, independently for each occurrence,

R₁ is aryl;

R₂ is hydrogen, alkyl, aryl, or aralkyl;

R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;

provided that when R₂ is hydrogen; R₃ is hydrogen; and R₄ is —C(═O)NHCH(CH₃)Ph; R₁ is not

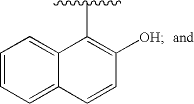

the compound is achiral or, when chiral, is a single stereoisomer or a mixture of stereoisomers.

In another aspect, a sirtuin inhibitory compound may be a compound of formula XI:

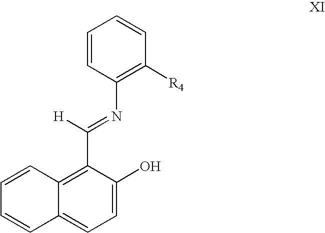

wherein, independently for each occurrence,

R₄ is —C(═O)ORₐ, —C(═O)N(Rₐ)₂ or —CN;

Rₐ is hydrogen, alkyl, aryl, or aralkyl;

provided that R₄ is not —C(═O)NHCH(CH₃)Ph; and

the compound is achiral or, when chiral, is a single stereoisomer or a mixture of stereoisomers.

In certain embodiments, sirtuin inhibitory compounds are represented by X or XI and the attendant definitions, wherein R₄ is —C(═O)OEt.

In certain embodiments, sirtuin inhibitory compounds are represented by X or XI and the attendant definitions, wherein R₄ is —C(═O)OH.

In certain embodiments, sirtuin inhibitory compounds are represented by X or XI and the attendant definitions, wherein R₄ is —C(═O)NH₂.

In certain embodiments, sirtuin inhibitory compounds are represented by X or XI and the attendant definitions, wherein R₄ is —CN.

Also included are pharmaceutically acceptable addition salts and complexes of the compounds of formula I-X. In cases wherein the compounds may have one or more chiral centers, unless specified, the compounds contemplated herein may be a single stereoisomer or racemic mixtures of stereoisomers.

In cases in which the sirtuin inhibitory compounds have unsaturated carbon-carbon double bonds, both the cis (Z) and trans (E) isomers are contemplated herein. In cases wherein the compounds may exist in tautomeric forms, such as keto-enol tautomers, such as

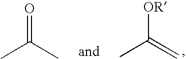

each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Also included in the methods presented herein are prodrugs of the sirtuin inhibitory compounds of formulas I-XI. Prodrugs are considered to be any covalently bonded carriers that release the active parent drug in vivo. Metabolites, e.g., degradation products are also included.

In certain embodiments, a compound is included within the generic structures of formula I-XI with the proviso that the compound is not a particular compound, such as a naturally occurring compound or that the compound is not present in a particular form, such as a naturally-occurring form.

A compound may have a binding affinity for a sirtuin of about 10⁻⁹M, 10⁻¹⁰M, 10⁻¹¹M, 10⁻¹²M or less. A compound may have an EC₅₀ for inhibiting the deacetylase activity of a sirtuin of less than about 1 nM, less than about 10 nM, less than about 100 nM, less than about 1 μM, less than about 10 μM, less than about 100 μM, or from about 1-10 nM, from about 10-100 nM, from about 0.1-1 μM, from about 1-10 μM or from about 10-100 μM. A compound may inhibit the deacetylase activity of a sirtuin by a factor of at least about 50%, 2, 5, 10, 20, 30, 50, or 100, as measured in an acellular assay or in a cell based assay, such as described in the Examples. A compound may cause at least a 10%, 30%, 50%, 80%, 2 fold, 5 fold, 10 fold, 50 fold or 100 fold greater inhibition of the deacetylase activity of SIRT1 relative to the same concentration of sirtinol or other compound described herein.

The effect of a compound on the activity of a sirtuin, such as SIRT1, may be determined as described, e.g., in Howitz et al. (Nature 425:191 (2003)) or as follows. For instance, sirtuin proteins may be contacted with a compound in vitro, e.g., in a solution, in a cell or in a cell extract. In one embodiment, a sirtuin protein is contacted with a compound in a solution and an activity of the sirtuin, e.g., its ability to deacetylate a protein, such as a histone or p53 or portion thereof, is determined. Generally a sirtuin is inhibited by a compound when at least one of its biological activities, e.g., deacetylation activity, is lower in the presence of a compound than in its absence. Inhibition may be by a factor of at least about 10%, 30%, 50%, 100% (i.e., a factor of two), 3, 10, 30, or 100.

A compound may inhibit more efficiently one sirtuin relative to one or more other sirtuins. For example, a compound may inhibit more efficiently hSirt1 (SIRT1) than the other sirtuins from the same species, e.g., hSIR2-7. A compound may inhibit more efficiently a sirtuin from a particular species relative to the homologous sirtuin from another species. For example, a compound may inhibit more efficiently a sirtuin from a microorganism, such as a pathogen, relative to the same sirtuin from humans. A sirtuin inhibiting compound may be more efficient in inhibiting one sirtuin relative to another by a factor of at least about 50%, 2 fold, 5 fold, 10, fold, 20 fold, 50 fold, or 100 fold.

A compound may traverse the cytoplasmic membrane of a cell. For example, a compound may have a cell-permeability of at least about 20%, 50%, 75%, 80%, 90% or 95%. A compound having a cell-permeability of at least about 20% means that at least about 20% of these compounds will enter a cell within a certain time frame when a given amount of these compounds is contacted with the cell.

A compound may have a normal half-life under normal atmospheric conditions of at least about 30 days, 60 days, 120 days, 6 months, or 1 year. One compound may be more stable in solution than another compound, e.g., sirtinol, by a factor of at least about 50%, 2 fold, 5 fold, 10 fold, 30 fold, 50 fold, or 100 fold.

### Exemplary Methods

Methods provided herein may include contacting a sirtuin or a cell comprising a sirtuin with a sirtuin inhibitory compound, such as those described herein. The sirtuin may be a eukaryotic, prokaryotic or plant sirtuin. The sirtuin may be from a vertebrate, e.g., a mammal, a fish, a reptile and amphibian. Mammalian sirtuins include those from humans, non-human primates, ovines, bovines, porcines, equines, canines, felines, sheep, rats and mice. In a preferred embodiment, the sirtuin is a human sirtuin, e.g., SIRT1, or a yeast sirtuin, e.g., Sir2. A sirtuin and an inhibitory compound may be contacted in solution. Alternatively, a sirtuin and an inhibitory compound are contacted in silico. In some embodiments, an inhibitory compound is contacted with a fragment of a sirtuin, e.g., a biologically active fragment.

When contacting an inhibitory compound with a sirtuin in a cell, the compound may be administered to the cell or the cell may be contacted with the compound. This is generally referred to as “treating a cell with a compound.” The cell may be a eukaryotic cell, e.g., a mammalian cell, such as a human cell, a yeast cell, a non-human primate cell, a bovine cell, an ovine cell, an equine cell, a porcine cell, a sheep cell, a bird (e.g., chicken or fowl) cell, a canine cell, a feline cell or a rodent (mouse or rat) cell. It can also be a non-mammalian cell, e.g., a fish cell. Yeast cells include S. cerevesiae and C. albicans. The cell may also be a prokaryotic cell, e.g., a bacterial cell. The cell may also be a single-cell microorganism, e.g., a protozoan. The cell may also be a metazoan cell, a plant cell or an insect cell. The application of the methods described herein to a large number of cell types is based at least on the high conservation of sirtuins from humans to fungi, protozoans, metazoans and plants.

A cell may be contacted with a solution having a concentration of an inhibitory compound of less than about 0.1 μM; 0.5 μM; less than about 1 μM; less than about 10 μM or less than about 100 μM. The concentration of the inhibitory compound may also be in the range of about 0.1 to 1 μM, about 1 to 10 μM or about 10 to 100 μM. The appropriate concentration may depend on the particular compound and the particular cell used as well as the desired effect. For example, a cell may be contacted with a “sirtuin inhibiting” concentration of an inhibitory compound, e.g., a concentration sufficient for inhibiting the sirtuin by a factor of at least about 10%, 30%, 50%, 100%, 3, 10, 30, or 100.

Contacting a cell with a sirtuin inhibitory compound may be used, e.g., to reduce the life span of the cell; to kill the cell, e.g., by apopotis; or to render the cell more susceptible to certain stresses, e.g., heatshock, radioactivity, osmotic stress, DNA damage, e.g., from UV, and chemotherapeutic drugs.

In one embodiment, a cell is in vitro. The cell may be from a cell line, in a primary cell culture (i.e., cells obtained from an organism, e.g., a human), or it may be an embryonic stem (ES) cell or progeny thereof. Contacting cells in vitro with a sirtuin inhibiting compound may be used, e.g., to identify conditions or drugs that induce a stress on the cell.

In certain embodiments, a cell is contacted with an inhibiting compound in vivo, such as in a subject. A method may comprise administering to a subject, e.g., a subject in need thereof, a therapeutically effective amount of a sirtuin inhibitor. Treatment may be prophylactic or therapeutic. The subject may be a human, a non-human primate, a bovine, an ovine, an equine, a porcine, a sheep, a canine, a feline or a rodent (mouse or rat). For example, an inhibiting compound may be administered to a subject. Administration may be local, e.g., topical, parenteral, oral, or other depending on the desired result of the administration (as further described herein). In an exemplary embodiment, cells are treated in vivo to decrease their life span; kill them and/or sensitize them against certain types of stresses. Administration may be followed by measuring a factor in the subject or in the cell, such as the activity of the sirtuin, lifespan or stress resistance. In an illustrative embodiment, a cell is obtained from a subject following administration of an inhibitory compound to the subject, such as by obtaining a biopsy, and the activity of the sirtuin is determined in the biopsy. The cell may be any cell of the subject, but in cases in which an inhibitory compound is administered locally, the cell is preferably a cell that is located in the vicinity of or at the site of administration.

Administration of a sirtuin inhibitory compound to a subject may be used to treat or prevent any disease or condition in which it is desirable to reduce the lifespan of a cell; kill a cell or render a cell more sensitive to certain stress conditions. Exemplary diseases or conditions include cancer, autoimmune diseases, inflammatory diseases, infectious diseases, asthma, allergic rhinitis (hay fever), and excema, or any other situation in which it is desirable to eliminate cells in a subject. Sirtuin inhibitory compounds or agents of the invention can be administered directly to the area containing the undesirable cells, e.g., in a tumor, such as in a cancer patient. These methods can also be used to eliminate cells or prevent further proliferation of undesirable cells of non-malignant tumors, e.g., warts, beauty spots, and fibromas. For example, a sirtuin inhibitory compound can be injected into a wart, or alternatively be included in a pharmaceutical composition for applying onto the wart. The methods may also be used to sensitize tumor cells to agents that rely on killing them, e.g., chemotherapeutic drugs.

A subject in need of therapy may be a subject having been diagnosed with a disease, e.g., cancer or an autoimmune disease. A subject may also be a subject who has been determined as being likely to develop cancer or an autoimmune disease, e.g., a subject having a gene indicating susceptibility of developing the disease, or a subject in whose family the disease is more frequent than normal.

In another embodiment, cells can be obtained from a subject, e.g., a human or other mammal, treated ex vivo according to the methods of the invention to remove undesirable cells, e.g., cancer cells, and then administered to the same or a different subject. Accordingly, cells or tissues may be obtained from a donor, treated ex vivo as described herein and administered to the same or different subject. In certain embodiments, cells are incubated with a sirtuin inhibitor in the presence or a drug, e.g., a chemotherapeutic drug, such as to increase the susceptibility of the cells to the effect of the drug. An exemplary situation in which one may use this method is in purging bone marrow (or blood) obtained from a cancer patient from cancer cells. A method may also be used to purge bone marrow or blood from autoimmune cells.

Exemplary cancers are those of the brain including glioblastomas, medulloblastoma, astrocytoma, oligodendroglioma, ependymomas; kidney; colon; lung; liver; pancreas; endometrium; spleen; small intestine; stomach; skin; head and neck; esophagus; hormone-dependent cancers including breast, prostate, testicular, and ovarian cancers; lymphomas (lymph node); and leukemias including cancer of blood cells and bone marrow. In cancers associated with solid tumors, an inhibitory compound may be administered directly into the tumor. Cancer of blood cells, e.g., leukemia can be treated by administering an inhibitory compound into the blood stream or into the bone marrow. Benign cell growth can also be treated, e.g., warts.

The compositions and methods described herein may also be used for the treatment of neoplasia disorders selected from the group consisting of acral lentiginous melanoma, actinic keratoses, adenocarcinoma, adenoid cystic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondrosarcoma, choroid plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiatied carcinoma, uveal melanoma, verrucous carcinoma, vipoma, well differentiated carcinoma, and Wilm's tumor.

In the context of reducing unwanted cell proliferation, and reducing tumor mass, an effective amount of an agent that inhibits a sirtuin may be an amount that reduces the level and/or rate of cell proliferation and/or reduces tumor mass by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 85%, or at least about 90%, or more, compared to the level and/or rate of cell proliferation and/or tumor mass in the absence of treatment with an agent that inhibits a sirtuin.

“Autoimmune disease” refers to a condition, which is characterized by a specific humoral or cell-mediated immune response against constituents of the body's own tissues, such as self-antigens or auto-antigens. Examples of autoimmune diseases that may be treated as described herein include but are not limited to Active Chronic Hepatitis, Addison's Disease, Anti-phospholipid Syndrome, Atopic Allergy, Autoimmune Atrophic Gastritis, Achlorhydra Autoimmune, Celiac Disease, Crohn's Disease, Cushing's Syndrome, Dermatomyositis, Diabetes (type I), Discoid Lupus, Erythematosis, Goodpasture's Syndrome, Grave's Disease, Hashimoto's Thyroiditis, Idiopathic Adrenal Atrophy, Idiopathic Thrombocytopenia, Insulin-dependent Diabetes, Lambert-Eaton Syndrome, Lupoid Hepatitis, some cases of Lymphopenia, Mixed Connective Tissue Disease, Multiple Sclerosis, Pemphigoid, Pemphigus Vulgaris, Pernicious Anema, Phacogenic Uveitis, Polyarteritis Nodosa, Polyglandular Auto. Syndromes, Primary Biliary Cirrhosis, Primary Sclerosing Cholangitis, Psoriasis, Raynaud's Syndrome, Reiter's Syndrome, Relapsing Polychondritis, Rheumatoid Arthritis, Schmidt's Syndrome, Limited Scleroderma (or CREST Syndrome), Severe Combined Immunodeficiency Syndrome (SCID), Sjogren's Syndrome, Sympathetic Ophthalmia, Systemic Lupus Erythematosis, Takayasu's Arteritis, Temporal Arteritis, Thyrotoxicosis, Type B Insulin Resistance, Ulcerative Colitis and Wegener's Granulomatosis, in which it is desirable to eliminate autoimmune cells. Viral infections such as herpes, HIV, adenovirus, and HTLV-1 associated malignant and benign disorders can also be treated by administration of compounds.

A sirtuin inhibitory compound may also be used to promote or increase weight gain (i.e., fat content) of cachexic patients, wherein Sirt1 mobilizes and depletes fat. A method may comprise identifying a subject as being underweight, at risk for weight loss or cachexia using clinical criteria, or being cachexic; and administering an effective amount of an agent that decreases SIRT1 activity to the subject. Other diseases in which sirtuin inhibiting compounds could be administered to stimulate weight gain include anorexia nervosa, wasting, and AIDS-related weight loss. They may also be used to lower blood glucose levels in subjects.

Whether in vitro, ex vivo, or in vivo, a cell may also be contacted with more than one compound. A cell may be contacted with at least 2, 3, 5, or 10 different compounds. A cell may be contacted simultaneously or sequentially with different compounds. In one embodiment, a sirtuin inhibitory compound may be administered as part of a combination therapy with another therapeutic agent. In an exemplary embodiment, the second therapeutic agent is another sirtuin inhibitory compound, such as nicotinamide or sirtinol, and/or an agent that kills cells. Such combination therapies may be administered simultaneously (e.g., more than one therapeutic agent administered at the same time) or sequentially (e.g., different therapeutic agents administered at different times during a treatment regimen).

Chemotherapeutic agents that may be coadministered with compounds described herein include: aminoglutethimide, amsacrine, anastrozole, asparaginase, bcg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imafinib, interferon, irinotecan, irinotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, ocreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic agents may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disrupters such as taxane (paclitaxel, docetaxel), vincristine, vinblastin, nocodazole, epothilones and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, cytoxin, dactinomycin, daunorubicin, docetaxel, doxorubicin, epirubicin, hexamethylmelamineoxaliplatin, iphosphamide, melphalan, mechlorethamine, mitomycin, mitoxantrone, nitrosourea, paclitaxel, plicamycin, procarbazine, teniposide, triethiylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors, epidermal growth factor (EGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; chromatin disrupters.

These chemotherapeutic agents may be used by themselves with a compound described herein as inducing cell death or reducing lifespan or increasing sensitivity to stress and/or in combination with other chemotherapeutics agents.

In addition to conventional chemotherapeutics, the compounds described herein can also be used with antisense RNA, RNAi or other polynucleotides to inhibit the expression of the cellular components that contribute to unwanted cellular proliferation that are targets of conventional chemotherapy. Such targets are, merely to illustrate, growth factors, growth factor receptors, cell cycle regulatory proteins, transcription factors, or signal transduction kinases.

The methods may be advantageous over combination therapies known in the art because it allows conventional chemotherapeutic agent to exert greater effect at lower dosage. In a preferred embodiment, the effective dose (ED₅₀) for a chemotherapeutic agent or combination of conventional chemotherapeutic agents when used in combination with a compound described herein is at least about 2 fold less than the ED₅₀ for the chemotherapeutic agent alone, and even more preferably at least about 5 fold, 10 fold or even 25 fold less. Conversely, the therapeutic index (TI) for such chemotherapeutic agent or combination of such chemotherapeutic agent when used in combination with a compound described herein can be at least about 2 fold greater than the TI for conventional chemotherapeutic regimen alone, and even more preferably at least about 5 fold, 10 fold or even 25 fold greater.

Dominant negative mutants of SIRT1 and a Sirt1 knockout mouse have shown that a defective SIRT1 protein increases sensitivity to radiation-induced apoptosis (Vaziri et al. (2001) Cell 107:149 and Cheng et al. (2003) PNAS 100:10794). Thus, inhibitors of SIRT1 may be used to increase the sensitivity of cells to certain cell death inducing conditions or agents, such as radiation. SIRT1 deacetylates the p65 nuclear factor kB (NF-kB) subunit, thereby impairing NF-kB driven transactivation of genes, such as pro-survival genes Bcl-XL, TRAF2 and cIAP2. Thus, SIRT1 inhibitors may be used to stimulate NF-kB mediated gene transcription.

Based at least in part on the fact that SIRT2 deacetylates tubulin, inhibitors of SIRT2 may be used for inhibiting tubulin deacetylation (North et al. (2003) Mol Cell 11:437 and U.S. Patent Application No. 20040028607). The microtubule network is formed by the polymerization of α/β tubulin heterodimers and plays an important role in the regulation of cell shape, intracellular transport, cell motility, and cell division. α and β tubulin sub-units are subject to numerous post translational modifications including tyrosination, phosphorylation, polyglutamylation, polyglycylation and acetylation. Tubulin represents one of the major acetylated cytoplasmic proteins. Acetylation of tubulin takes place on lysine-40 of α-tubulin, which based on the crystal structure of the tubulin heterodimer, is predicted to lie within the luminal side of the polymerized microtubule. Human SIRT2 removes an acetyl group from lysine-40 of α-tubulin. Deacetylation of tubulin also results in a reduction in the specific interaction of tubulin with the transcription factor myc-interacting zinc finger-1 (“MIZ-1”; GenBank Accession No. Q13105). Thus, SIRT2 inhibitors, such as those disclosed herein, may be used to inhibit cellular proliferation, e.g., in cancer, to stabilize microtubules and to modulate cell structure and motility.

SIRT2 also binds and deacetylates the MyoD transcription factor and impairs MyoD-driven gene expression and muscle differentiation (Fulco et al. (2003) Mol. Cell 12:51. Thus, SIRT2 inhibitors may be used to stimulate or preserve muscle differentiation and thereby treat or prevent diseases that may benefit from stimulation or preservation of muscle differentiation or muscle regeneration, such as a disease or condition associated with degeneration of muscle tissue, muscular atrophy, muscular dystrophy, and muscular cachexia. Muscle differentiation may also be triggered in vitro, such as on progenitor or myoblast cells.

SIRT2 is responsible for the delay in mitotic exit. Overexpression of SIRT2 causes cells to delay reentry into the cell cycle, whereas this is not observed with overexpression of a deacetylase deficient SIRT2 mutant (Dryden et al. (2003) Mol. Cell. Biol. 23:3173). It has also been shown that SIRT2 delays or blocks starfish oocyte maturation (Borra et al. (2002) J. Biol. Chem. 277:12632). Based at least on these observations, SIRT2 inhibitors may be used to modulate, in particular, to accelerate mitotic exit and reentry into the cell cycle.

Since SIRT2 also interacts with the homeobox transcription factor HOXA10, modulating SIRT2 may affect mammalian development (Bae et al. (2004) J. Biochem. 135:695.

SIRT2 may be inhibited with compounds having formula I, III, IV, VI, VII or IX, such as those set forth in Example 11. A SIRT2 inhibitory compound may also be a compound having a formula selected from the group of formulas I, III, IV, VI, VII or IX, with the proviso that the compound is not a specific compound covered by one of the formulas, such as sirtinol.

The compounds described herein could also inhibit the other members of the sirtuin deacetylase family (e.g., SIRT3-7), provided that they have a deacetalating activity.

Sirtuin inhibitory compounds may also be used for reducing the lifespan of microorganisms, such as prokaryotes; for killing the microorganisms or for rendering them more sensitive to stress. In one embodiment, a method for treating a subject having an infection by a microorganism comprises administering to the subject a therapeutically effective amount of a sirtuin inhibitory compound. A pharmaceutical composition comprising a sirtuin inhibitory compound can be applied to the location of the infection.

Sirtuin inhibitory compounds may be used to debilitate the defense of disease agents (e.g., pathogens that have sirtuins) or for altering their metabolism. For example, parasites of the Leishmania sp. are less viable if Sir2 is inhibited. Accordingly, a composition comprising a sirtuin inhibitory compound may be administered to a subject for treating an infection by a microorganism expressing a sirtuin, including, but not limited to Archaeglobus fulgidus, Bacillus antliracis, Bacteriodes fragilis, Bifidobacterium longum, Bordetella pertussis, Burkholderia pseudomallei, Clostridium acetobutylium, Corynebacterium glutamicum, Escherichia coli, Enteroccus faecalis, Eremothecium gossypii, Erythrobacter litoralis, Helicobacter hepaticies, Helicobacter pylori, Leishmania sp., Listeria monocytogenes, Mycobacterium tuberculosis, Plasmodium falciparum, Pseudomonas aeruginosa, Salmonella typhimerium, Salmonella enterica, Staphylococcus aureus, Streptococcus pneumoniae, Streptomyces coelicolor, Streptomyces pyogenes, Sinorhizobium meliloti, Thermotoga maritma, Vibrio cholerae, Vibrio vulnificus, Yarrowia lipolytica, and Yersinia pestis.

Other microorganisms that may be killed or sensitized to stress include yeast, such as pathogenic yeast strains and fungus expressing a sirtuin. In one embodiment, a subject having a yeast infection is treated with a therapeutically effective amount of a sirtuin inhibitor. Yeast and fungi may include but are not limited to Candida albicans, Saccharomyces cerevisiae, Schizosaccharomyces pombe, Cryptococcus neoformans, Blastomyces dermatitidis, Microsporum canis, and Microsporum gypsum. In an exemplary embodiment, a subject infected with Candida albicans is treated with a therapeutically effective amount of a sirtuin inhibitor to reduce skin infections, enteritis, vaginitis, thrush, and/or systemic disease. A subject infected with Cryptococcus neoforms may be treated with a therapeutically effective amount of a sirtuin inhibitor to reduce meningtitis, pulmonary and disseminated disease, skin and/or bone lesions. A subject infected with Blastomyces dermatitidis may be treated with a therapeutically effective amount of a sirtuin inhibitor to reduce chronic granulomatous and/or suppurative disease. A subject infected with Microsporum canis or Microsporum gypsum may be treated with a therapeutically effective amount of a sirtuin inhibitor to reduce Tinea Captitis, Tinea Corporis, and/or Tinea Barbae.

Compounds for treating pathogenic microorganisms will preferably inactivate the microorganism's sirtuin relative to the human sirtuin. A sirtuin inhibitory compound may inactivate a microorganism's sirtuin at least about 2 fold, 5 fold, 10 fold, 20 fold, 50 fold, or 100 fold more efficiently relative to one or more human sirtuins. A sirtuin inhibitory compound may bind preferentially to the sirtuins expressed by the microorganism relative to human sirtuins. For example, a sirtuin inhibitory compound may have a higher affinity for the sirtuin from the microorganism relative to the sirtuin from humans.

Methods described herein may also be used to reduce the lifespan, kill or sensitize microorganisms or other organisms that are pathogens for non-human animals or plants, as well as those microorganisms that cause undesirable consequences in the environment, such as mildew.

Methods described herein may also be used in agriculture to reduce the lifespan, kill or sensitize undesirable plants (e.g., weeds) to infection resulting in the killing of the undesirable plant.

### Pharmaceutical Formulations

Pharmaceutical compositions for use in accordance with the present methods may be formulated in conventional manner using one or more physiologically acceptable carriers or excipients. Thus, activating compounds and their physiologically acceptable salts and solvates may be formulated for administration by, for example, injection, inhalation or insufflation (either through the mouth or the nose) or oral, buccal, parenteral or rectal administration. In one embodiment, the compound is administered locally, at the site where the target cells, e.g., diseased cells, are present, i.e., in the blood or in a joint.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

Pharmaceutical compositions (including cosmetic preparations) may comprise from about 0.00001 to 100% such as from 0.001 to 10% or from 0.1% to 5% by weight of one or more compounds described herein. In certain topical formulations, the active compound is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Compounds described herein may be stored in oxygen free environment according to methods in the art. For example, sirtinol analogues can be prepared in an airtight capsule for oral administration, such as Capsugel from Pfizer, Inc.

Cells, e.g., treated ex vivo with a compound described herein, can be administered according to methods for administering a graft to a subject, which may be accompanied, e.g., by administration of an immunosuppressant drug, e.g., cyclosporin A. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000.

Administration of a sirtuin inhibitor or other agent that decreases the activity or protein level of a sirtuin may be followed by measuring a factor in the subject, such as measuring the activity of the sirtuin. In an illustrative embodiment, a cell is obtained from a subject following administration of an inhibitory compound to the subject, such as by obtaining a biopsy, and the activity of the sirtuin or sirtuin expression level is determined in the biopsy. Alternatively, biomarkers, such as plasma biomarkers may be followed. The cell may be any cell of the subject, but in cases in which an inhibitory compound is administered locally, the cell is preferably a cell that is located in the vicinity of the site of administration.

### Screening Methods

Also provided herein are methods for identifying sirtuin inhibitors. Methods may test the ability of a compound having a similar structure to the structures described herein to inhibit a sirtuin.

In one embodiment, a method is a computer assisted method. An exemplary method may comprise (i) supplying a computer modeling application with a set of structure coordinates of a molecule or molecular complex, the molecule or molecular complex including at least a portion of a sirtuin; (ii) supplying the computer modeling application with a set of structure coordinates of a chemical entity; and (iii) determining whether the chemical entity is an inhibitor expected to bind to or interfere with the molecule or molecular complex, wherein binding to or interfering with the molecule or molecular complex is indicative of potential inhibition of the activity of the sirtuin. The chemical entity may be an analog of a sirtuin inhibitor disclosed herein. A method may comprise providing the structure of a compound that is similar to any of the structures described herein, and testing its ability to bind to a sirtuin as described in the Examples.

Another method for identifying an inhibitor of the activity of a sirtuin comprises: (i) contacting a sirtuin or fragment or domain thereof with a test compound for a time sufficient for the test compound to potentially bind to the sirtuin or fragment or domain thereof; and (ii) determining the activity of protein; wherein a lower activity of the protein in the presence of the test compound relative to the absence of the test compound indicates that the test compound is an inhibitor of the sirtuin.

Inhibitors of sirtuins may also be identified and developed as set forth below and otherwise using techniques and methods known to those of skill in the art. In certain embodiments, methods may test the ability of a compound having a similar structure to the structures described herein to inhibit a sirtuin.

Such novel sirtuin inhibitors may be employed, for instance, as described for the inhibitors of the invention described herein, and may be used in the manufacture of a medicament for any number of uses as described herein.

(a) Drug Design

A number of techniques can be used to screen, identify, select and design chemical entities capable of associating with sirtuins. Knowledge of the structure for a sirtuin, determined in accordance with the methods described herein, permits the design and/or identification of molecules and/or other inhibitors which have a shape complementary to the conformation of a sirtuin, or more particularly, a druggable region thereof.

Exemplary sirtuin druggable regions include, but are not limited to, the Ac—K substrate binding site, the NAD+ substrate binding site, non-competitive side pocket that blocks nicotinamide escape and entrance channel of the acetylated lysine substrate.

It is understood that such techniques and methods may use, in addition to the exact structural coordinates and other information for a sirtuin, structural equivalents thereof described herein (including, for example, those structural coordinates that are derived from the structural coordinates of amino acids contained in a druggable region as described herein).

In one aspect, the method of drug design generally includes computationally evaluating the potential of a selected chemical entity to associate with any of the molecules or complexes of the present invention (or portions thereof). For example, this method may include the steps of (a) employing computational means to perform a fitting operation between the selected chemical entity and a druggable region of the molecule or complex; and (b) analyzing the results of said fitting operation to quantify the association between the chemical entity and the druggable region.

A chemical entity may be examined either through visual inspection or through the use of computer modeling using a docking program such as GRAM, DOCK, or AUTODOCK (Dunbrack et al., Folding & Design, 2:27-42 (1997)). This procedure can include computer fitting of chemical entities to a target to ascertain how well the shape and the chemical structure of each chemical entity will complement or interfere with the structure of the subject polypeptide (Bugg et al., Scientific American, Dec.: 92-98 (1993); West et al., TIPS, 16:67-74 (1995)). Computer programs may also be employed to estimate the attraction, repulsion, and steric hindrance of the chemical entity to a druggable region, for example. Generally, the tighter the fit (e.g., the lower the steric hindrance, and/or the greater the attractive force) the more potent the chemical entity will be because these properties are consistent with a tighter binding constant. Furthermore, the more specificity in the design of a chemical entity the more likely that the chemical entity will not interfere with related proteins, which may minimize potential side-effects due to unwanted interactions.

A variety of computational methods for molecular design, in which the steric and electronic properties of druggable regions are used to guide the design of chemical entities, are known: Cohen et al. (1990) J. Med. Cam. 33: 883-894; Kuntz et al. (1982) J. Mol. Biol. 161: 269-288; DesJarlais (1988) J. Med. Cain. 31: 722-729; Bartlett et al. (1989) Spec. Publ., Roy. Soc. Chem. 78: 182-196; Goodford et al. (1985) J. Med. Cam. 28: 849-857; and DesJarlais et al. J. Med. Cam. 29: 2149-2153. Directed methods generally fall into two categories: (1) design by analogy in which 3-D structures of known chemical entities (such as from a crystallographic database) are docked to the druggable region and scored for goodness-of-fit; and (2) de novo design, in which the chemical entity is constructed piece-wise in the druggable region. The chemical entity may be screened as part of a library or a database of molecules. Databases which may be used include ACD (Molecular Designs Limited), NCI (National Cancer Institute), CCDC (Cambridge Crystallographic Data Center), CAST (Chemical Abstract Service), Derwent (Derwent Information Limited), Maybridge (Maybridge Chemical Company Ltd), Aldrich (Aldrich Chemical Company), DOCK (University of California in San Francisco), and the Directory of Natural Products (Chapman & Hall). Computer programs such as CONCORD (Tripos Associates) or DB-Converter (Molecular Simulations Limited) can be used to convert a data set represented in two dimensions to one represented in three dimensions.

Chemical entities may be tested for their capacity to fit spatially with a druggable region or other portion of a target protein. As used herein, the term “fits spatially” means that the three-dimensional structure of the chemical entity is accommodated geometrically by a druggable region. A favorable geometric fit occurs when the surface area of the chemical entity is in close proximity with the surface area of the druggable region without forming unfavorable interactions. A favorable complementary interaction occurs where the chemical entity interacts by hydrophobic, aromatic, ionic, dipolar, or hydrogen donating and accepting forces. Unfavorable interactions may be steric hindrance between atoms in the chemical entity and atoms in the druggable region.

If a model is a computer model, the chemical entities may be positioned in a druggable region through computational docking. If, on the other hand, the model is a structural model, the chemical entities may be positioned in the druggable region by, for example, manual docking.

In an illustrative embodiment, the design of a potential inhibitor begins from the general perspective of shape complimentary for the druggable region of a sirtuin, and a search algorithm is employed which is capable of scanning a database of small molecules of known three-dimensional structure for chemical entities which fit geometrically with the target druggable region. Most algorithms of this type provide a method for finding a wide assortment of chemical entities that are complementary to the shape of a druggable region of the subject sirtuin. Each of a set of chemical entities from a particular data-base, such as the Cambridge Crystallographic Data Bank (CCDB) (Allen et al. (1973) J. Chem. Doc. 13: 119), is individually docked to the druggable region of a sirtuin in a number of geometrically permissible orientations with use of a docking algorithm. In certain embodiments, a set of computer algorithms called DOCK, can be used to characterize the shape of invaginations and grooves that form the active sites and recognition surfaces of the druggable region (Kuntz et al. (1982) J. Mol. Biol 161: 269-288). The program can also search a database of small molecules for templates whose shapes are complementary to particular binding sites of a sirtuin (DesJarlais et al. (1988) J Med Chem 31: 722-729).

The orientations are evaluated for goodness-of-fit and the best are kept for further examination using molecular mechanics programs, such as AMBER or CHARMM. Such algorithms have previously proven successful in finding a variety of chemical entities that are complementary in shape to a druggable region.

Goodford (1985, J Med Chem 28:849-857) and Boobbyer et al. (1989, J Med Chem 32:1083-1094) have produced a computer program (GRID) which seeks to determine regions of high affinity for different chemical groups (termed probes) of the druggable region. GRID hence provides a tool for suggesting modifications to known chemical entities that might enhance binding. It may be anticipated that some of the sites discerned by GRID as regions of high affinity correspond to “pharmacophoric patterns” determined inferentially from a series of known ligands. As used herein, a “pharmacophoric pattern” is a geometric arrangement of features of chemical entities that is believed to be important for binding. Attempts have been made to use pharmacophoric patterns as a search screen for novel ligands (Jakes et al. (1987) J Mol Graph 5:41-48; Brint et al. (1987) J Mol Graph 5:49-56; Jakes et al. (1986) J Mol Graph 4:12-20).

Yet a further embodiment of the present invention utilizes a computer algorithm such as CLIX which searches such databases as CCDB for chemical entities which can be oriented with the druggable region in a way that is both sterically acceptable and has a high likelihood of achieving favorable chemical interactions between the chemical entity and the surrounding amino acid residues. The method is based on characterizing the region in terms of an ensemble of favorable binding positions for different chemical groups and then searching for orientations of the chemical entities that cause maximum spatial coincidence of individual candidate chemical groups with members of the ensemble. The algorithmic details of CLIX is described in Lawrence et al. (1992) Proteins 12:31-41.

In this way, the efficiency with which a chemical entity may bind to or interfere with a druggable region may be tested and optimized by computational evaluation. For example, for a favorable association with a druggable region, a chemical entity must preferably demonstrate a relatively small difference in energy between its bound and fine states (i.e., a small deformation energy of binding). Thus, certain, more desirable chemical entities will be designed with a deformation energy of binding of not greater than about 10 kcal/mole, and more preferably, not greater than 7 kcal/mole. Chemical entities may interact with a druggable region in more than one conformation that is similar in overall binding energy. In those cases, the deformation energy of binding is taken to be the difference between the energy of the free entity and the average energy of the conformations observed when the chemical entity binds to the target.

In this way, the present invention provides computer-assisted methods for identifying or designing a potential inhibitor of sirtuin activity including: supplying a computer modeling application with a set of structure coordinates of a sirtuin or sirtuin complex, the sirtuin or sirtuin complex including at least a portion of a druggable region from a sirtuin; supplying the computer modeling application with a set of structure coordinates of a chemical entity; and determining whether the chemical entity is expected to bind to the sirtuin or sirtuin complex, wherein binding to the sirtuin or sirtuin complex is indicative of potential inhibition of the sirtuin.

In another aspect, the present invention provides a computer-assisted method for identifying or designing a potential sirtuin inhibitor, supplying a computer modeling application with a set of structure coordinates of a sirtuin or sirtuin complex, the sirtuin or sirtuin complex including at least a portion of a druggable region of a sirtuin; supplying the computer modeling application with a set of structure coordinates for a chemical entity; evaluating the potential binding interactions between the chemical entity and active site of the molecule or molecular complex; structurally modifying the chemical entity to yield a set of structure coordinates for a modified chemical entity, and determining whether the modified chemical entity is expected to bind to the sirtuin or sirtuin complex, wherein binding to the sirtuin or sirtuin complex is indicative of potential sirtuin inhibitor.

In other embodiments, a potential inhibitor can be obtained by screening a chemical or peptide library (Scott and Smith, Science, 249:386-390 (1990); Cwirla et al., Proc. Natl. Acad. Sci., 87:6378-6382 (1990); Devlin et al., Science, 249:404-406 (1990)). A potential inhibitor selected in this manner could then be systematically modified by computer modeling programs until one or more promising potential drugs are identified. Such analysis has been shown to be effective in the development of HIV protease inhibitors (Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1:109-128 (1993)). Alternatively, the potential inhibitor may be synthesized de novo, for example, using modifications of the methods described herein.

For example, in certain embodiments, the present invention provides a method for making a potential sirtuin inhibitor, the method including synthesizing a chemical entity or a molecule containing the chemical entity to yield a potential sirtuin inhibitor, the chemical entity having been identified during a computer-assisted process including supplying a computer modeling application with a set of structure coordinates of a sirtuin or sirtuin complex, the sirtuin or sirtuin complex including at least one druggable region from a sirtuin; supplying the computer modeling application with a set of structure coordinates of a chemical entity; and determining whether the chemical entity is expected to bind to the sirtuin or sirtuin complex at the active site, wherein binding to the sirtuin or sirtuin complex is indicative of potential inhibition. This method may further include the steps of evaluating the potential binding interactions between the chemical entity and the active site or other druggable region of the sirtuin or sirtuin complex and structurally modifying the chemical entity to yield a set of structure coordinates for a modified chemical entity, which steps may be repeated one or more times.

Once a potential inhibitor is identified, it can then be tested in any assay for sirtuin activity, including those described herein, optionally as a high throughput assay. Further refinements to the structure of the inhibitor will generally be necessary and can be made by the successive iterations of any and/or all of the steps provided by the particular screening assay, in particular further structural analysis by e.g., ¹⁵N NMR relaxation rate determinations or x-ray crystallography with the modulator bound to the subject polypeptide. These studies may be performed in conjunction with biochemical assays.

Once identified, a potential inhibitor may be used as a model structure, and analogs to the compound can be obtained. The analogs are then screened for their ability to bind a sirtuin. An analog of the potential inhibitor might be chosen as an inhibitor when it binds to a sirtuin with a higher binding affinity than the predecessor inhibitor.

In a related approach, iterative drug design is used to identify sirtuin inhibitors. Iterative drug design is a method for optimizing associations between a protein and an inhibitor by determining and evaluating the three dimensional structures of successive sets of protein/inhibitor complexes. In iterative drug design, crystals of a series of protein/inhibitor complexes are obtained and then the three-dimensional structures of each complex is solved. Such an approach provides insight into the association between the proteins and inhibitors of each complex. For example, this approach may be accomplished by selecting compounds with inhibitory activity, obtaining crystals of this new sirtuin/compound complex, solving the three dimensional structure of the complex, and comparing the associations between the new complex and previously solved sirtuin/inhibitor complexes. By observing how changes in the inhibitor affected the sirtuin/inhibitor associations, these associations may be optimized.

In addition to designing and/or identifying a chemical entity to associate with a druggable region, as described above, the same techniques and methods may be used to design and/or identify chemical entities that either associate, or do not associate, with affinity regions, selectivity regions or undesired regions of sirtuins. By such methods, selectivity for one or a few targets, or alternatively for multiple targets, from the same species or from multiple species, can be achieved.

For example, a chemical entity may be designed and/or identified for which the binding energy for one druggable region, e.g., an affinity region or selectivity region, is more favorable than that for another region, e.g., an undesired region, by about 20%, 30%, 50% to about 60% or more. It may be the case that the difference is observed between (a) more than two regions, (b) between different regions (selectivity, affinity or undesirable) from the same target, (c) between regions of different targets, (d) between regions of homologs from different species, or (e) between other combinations. Alternatively, the comparison may be made by reference to the K_{d}, usually the apparent K_{d}, of said chemical entity with the two or more regions in question.

In another aspect, prospective inhibitors are screened for binding to two nearby druggable regions on a sirtuin. For example, a inhibitor that binds a first region of a sirtuin does not bind a second nearby region. Binding to the second region can be determined by monitoring changes in a different set of amide chemical shifts in either the original screen or a second screen conducted in the presence of an inhibitor (or potential inhibitor) for the first region. From an analysis of the chemical shift changes, the approximate location of a potential inhibitor for the second region is identified. Optimization of the second inhibitor for binding to the region is then carried out by screening structurally related compounds (e.g., analogs as described above). When inhibitors for the first region and the second region are identified, their location and orientation in the ternary complex can be determined experimentally. On the basis of this structural information, a linked compound, e.g., a consolidated inhibitor, is synthesized in which the inhibitor for the first region and the inhibitor for the second region are linked. In certain embodiments, the two inhibitors are covalently linked to form a consolidated inhibitor. This consolidated inhibitor may be tested to determine if it has a higher binding affinity for the target sirtuin than either of the two individual inhibitors. A consolidated inhibitor is selected as a inhibitor when it has a higher binding affinity for the target than either of the two inhibitors. Larger consolidated inhibitors can be constructed in an analogous manner, e.g., linking three inhibitors which bind to three nearby regions on the target to form a multilinked consolidated inhibitor that has an even higher affinity for the target than the linked inhibitor. In this example, it is assumed that is desirable to have the inhibitor bind to all the druggable regions. However, it may be the case that binding to certain of the druggable regions is not desirable, so that the same techniques may be used to identify inhibitors and consolidated inhibitors that show increased specificity based on binding to at least one but not all druggable regions of a target.

Provided herein, therefore, are a number of methods for identifying sirtuin inhibitors using drug design methods as described above. The chemical entity used in the methods may be an analog of a sirtuin inhibitors disclosed herein.

In one embodiment, a method for identifying a potential inhibitor of a sirtuin comprises: (a) providing the three-dimensional coordinates of a sirtuin or a portion thereof comprising a druggable region and (b) selecting from a database at least one compound that comprises three dimensional coordinates which indicate that the compound may bind the druggable region, wherein the selected compound is a potential inhibitor of a sirtuin. The selecting step may be done manually or via an algorithm. For example such a method may comprise: (a) supplying a computer modeling application with a set of structure coordinates of a sirtuin or sirtuin complex, the sirtuin or sirtuin complex including at least a portion of a sirtuin; (b) supplying the computer modeling application with a set of structure coordinates of a candidate chemical entity; and (c) determining whether the chemical entity is an inhibitor expected to bind to or interfere with the sirtuin or sirtuin complex, wherein binding to or interfering with the sirtuin or sirtuin complex is indicative of potential inhibition of the activity of the sirtuin. The determining step may include comprise performing a fitting operation between the chemical entity and a druggable region of the sirtuin or sirtuin complex, followed by computationally analyzing the results of the fitting operation to quantify the association between the chemical entity and the druggable region.

In another embodiment, a method for identifying a potential inhibitor of a sirtuin comprises: (a) providing the three-dimensional coordinates of a sirtuin or sirtuin complex, the sirtuin or sirtuin complex including at least a portion of a sirtuin; (b) optimizing the binding of a chemical entity to a druggable region of the sirtuin or sirtuin complex using an algorithm and (c) determining whether the chemical entity exhibits improved binding or interference with the formation of a sirtuin complex relative to known compounds. For example, such a method may comprise: (a) providing the three-dimensional coordinates of a sirtuin or sirtuin complex, the sirtuin or sirtuin complex including at least a portion of a sirtuin; (b) providing a set of structure coordinates for a chemical entity; (c) evaluating the potential binding interactions between the chemical entity and the sirtuin or sirtuin complex; (d) structurally modifying the chemical entity to yield a set of structure coordinates for a modified chemical entity; and (e) determining whether the modified chemical entity exhibits improved binding or interference with the formation of a sirtuin complex relative to known compounds.

In certain embodiments of the above methods, the chemical entities may be selected from a library or database of chemical entities. In other embodiments, the chemical entity may be computationally built de novo or by using a scaffold structure, e.g. such as the scaffold of the sirtuin inhibitors described herein. For example, a chemical entity may be designed to bind to a druggable region based on the three dimensional structure of the druggable region of a sirtuin.

In another aspect, a method for designing a sirtuin inhibitor comprises: (a) providing the three dimensional structure of a sirtuin or a fragment thereof; (b) synthesizing a potential inhibitor based on the three dimensional structure of the sirtuin or fragment; (c) contacting a sirtuin or fragment or domain thereof with the potential inhibitor; and (d) assaying sirtuin activity, wherein a change in sirtuin activity indicates that the compound may be a sirtuin inhibitor.

The methods may comprise providing the structure of a compound that is similar to any of the structures described herein, and testing its ability to bind to a sirtuin as described in the Examples.

(b) In Vitro Assays

Sirtuins may be used to assess the activity of small molecules and other inhibitors in in vitro assays. In one embodiment of such an assay, agents are identified which modulate the biological activity of a sirtuin, sirtuin interaction of interest or sirtuin complex. In certain embodiments, the test agent is a small organic molecule.

Assays may employ kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof.

The method of screening may involve high-throughput techniques. For example, to screen for inhibitors, a synthetic reaction mix, a cellular compartment, such as a membrane, cell envelope or cell wall, or a preparation of any thereof, comprising a sirtuin and a labeled substrate or ligand of the sirtuin is incubated in the absence or the presence of a candidate molecule that may be a sirtuin inhibitor. The ability of the candidate molecule to inhibit a sirtuin is reflected in decreased binding of the labeled ligand or decreased production of product from such substrate. Detection of the rate or level of production of product from substrate may be enhanced by using a reporter system. Reporter systems that may be useful in this regard include but are not limited to colorimetric labeled substrate converted into product, a reporter gene that is responsive to changes in sirtuin activity, and binding assays known in the art.

Another example of an assay for a sirtuin inhibitor is a competitive assay that combines a sirtuin and a potential inhibitor with molecules that bind to the sirtuin, recombinant molecules that bind to the sirtuin, natural substrates or ligands, or substrate or ligand mimetics, under appropriate conditions for a competitive inhibition assay. Sirtuins can be labeled, such as by radioactivity or a calorimetric compound, such that the number of molecules of sirtuin bound to a binding molecule or converted to product can be determined accurately to assess the effectiveness of the potential inhibitor.

A number of methods for identifying a molecule which inhibits sirtuin activity are described herein or are known in the art. For example, one such method for identifying an inhibitor of the activity of a sirtuin comprises: (i) contacting a sirtuin or fragment or domain thereof with a test compound for a time sufficient for the test compound to potentially bind to the sirtuin or fragment or domain thereof, and (ii) determining the activity of protein; wherein a lower activity of the protein in the presence of the test compound relative to the absence of the test compound indicates that the test compound is an inhibitor of the sirtuin.

Exemplary computer-assisted methods for identifying a sirtuin inhibitor comprise (a) supplying a computer modeling application with a set of three-dimensional structure coordinates so as to define part or all of a sirtuin or sirtuin complex; (b) supplying the computer modeling application with a set of structure coordinates of a chemical entity; and (c) determining whether the chemical entity is expected to bind to or interfere with the sirtuin or sirtuin complex. A computer-assisted method for identifying a sirtuin inhibitor may also comprise (a) supplying a computer modeling application with a set of three-dimensional structure coordinates so as to define part or all of a sirtuin or sirtuin complex; (b) supplying the computer modeling application with a set of structure coordinates for a chemical entity; (c) evaluating the potential binding interactions between the chemical entity and the sirtuin or sirtuin complex; (d) structurally modifying the chemical entity to yield a set of structure coordinates for a modified chemical entity; and (e) determining whether the modified chemical entity is an inhibitor expected to bind to or interfere with the sirtuin or sirtuin complex, wherein binding to or interfering with the sirtuin or sirtuin complex is indicative of a sirtuin inhibitor.

A computer-assisted method for designing a sirtuin inhibitor de novo may comprise (a) supplying a computer modeling application with a set of three-dimensional structure coordinates so as to define part or all of a sirtuin or sirtuin complex; (b) computationally building a chemical entity represented by a set of structure coordinates; and (c) determining whether the modified chemical entity is an inhibitor expected to bind to or interfere with the sirtuin or sirtuin complex, wherein binding to or interfering with the sirtuin or sirtuin complex is indicative of a sirtuin inhibitor. A method for identifying a potential modulator of a polypeptide from a database may comprise (a) providing a set of three-dimensional structure coordinates so as to define part or all of a sirtuin or sirtuin complex; (b) identifying a druggable region of the sirtuin or sirtuin complex; and (c) selecting from a database at least one potential inhibitor comprising three dimensional coordinates which indicate that the inhibitor may bind or interfere with the druggable region.

Any of the methods described herein may further comprise supplying or synthesizing a potential inhibitor, then assaying the potential inhibitor to determine whether it inhibits sirtuin activity. A method for identifying a sirtuin inhibitor may comprise (a) supplying a computer modeling application with a set of three-dimensional structure coordinates so as to define part or all of a sirtuin or sirtuin complex; (b) obtaining a potential inhibitor using the three dimensional structure; (c) contacting the potential inhibitor with a sirtuin; and (d) assaying the activity of the sirtuin, wherein a change in the activity of the sirtuin indicates that the compound may be useful as a sirtuin inhibitor.

Further provided herein are methods for preparing a potential inhibitor of a sirtuin, e.g., comprising (a) generating one or more three-dimensional structures of a molecule comprising a druggable region from a sirtuin; (b) employing one or more of the three dimensional structures of the molecule to design or select a potential inhibitor of the druggable region; and (c) synthesizing or obtaining the inhibitor. A method for making an inhibitor of sirtuin activity may also comprise chemically or enzymatically synthesizing a chemical entity to yield an inhibitor of sirtuin activity, the chemical entity having been identified during a computer-assisted process comprising supplying a computer modeling application with a set of structure coordinates of a sirtuin or sirtuin complex, the sirtuin or sirtuin complex comprising at least a portion of at least one druggable region; supplying the computer modeling application with a set of structure coordinates of a chemical entity; and determining whether the chemical entity is expected to bind or to interfere with the sirtuin or sirtuin complex at a druggable region, wherein binding to or interfering with the sirtuin or sirtuin complex is indicative of potential inhibition of sirtuin activity.

### Kits

Also provided herein are kits, e.g., kits for therapeutic purposes. A kit may comprise one or more inhibitory compounds described herein, e.g., in premeasured doses. A kit may optionally comprise devices for contacting cells with the compounds and instructions for use. Devices include syringes, stents and other devices for introducing a compound into a subject or applying it to the skin of a subject.

A kit may further contain one or more components or agents for measuring a factor, such as the activity and/or the expression level of a sirtuin, e.g., in tissue samples.

Kits for screening assays are also provided. Exemplary kits comprise one or more agents for conducting a screening assay, such as a sirtuin, or a biologically active portion thereof, or a cell or cell extract comprising such. Any of the kits may also comprise instructions for use.

### EXEMPLIFICATION

The invention having been generally described, may be more readily understood by reference to the following examples, which are included merely for purposes of illustration of certain aspects and embodiments of the present invention, and are not intended to limit the invention in any way. Some of the examples described herein are set forth in Mai et al. (2005) J. Med. Chem. 48, 7789, which is specifically incorporated by reference herein.

### Example 1

### Synthesis of Sirtinol Analogues

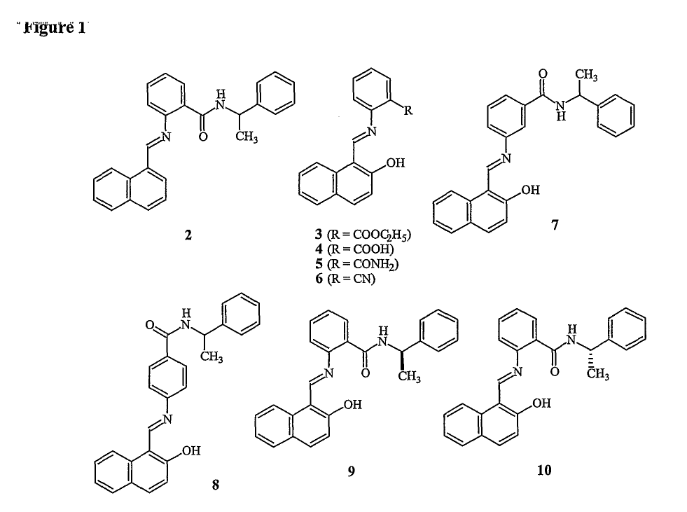

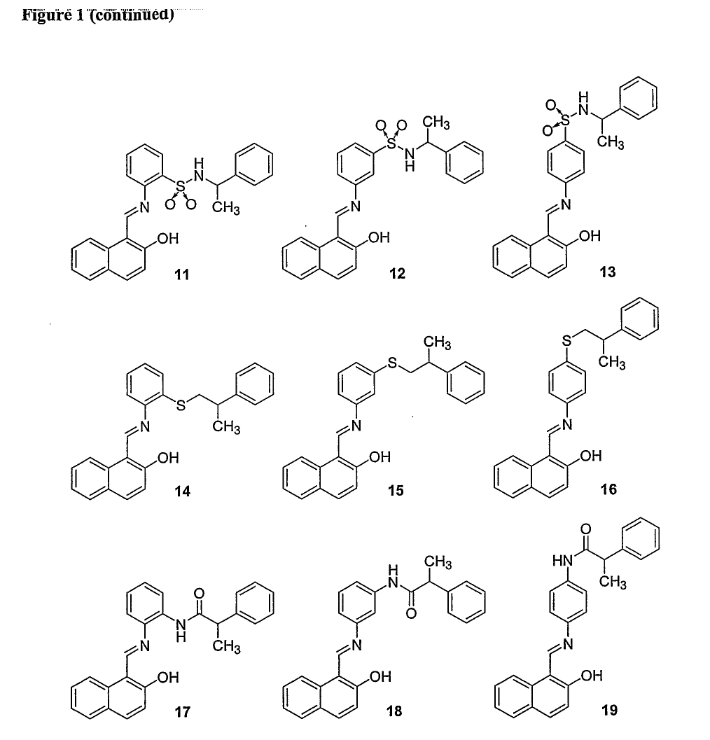

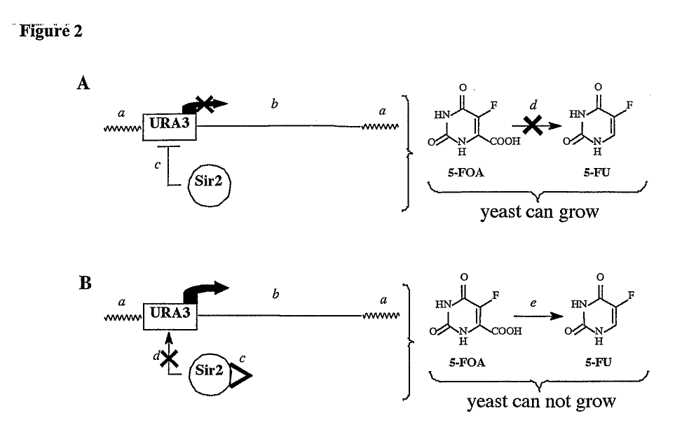

We prepared a number of 1 analogues by modification of the 2-hydroxynaphthyl group (compound 2) or the benzamide function (compounds 3-6) of the sirtinol structure. Two sirtinol isomers (meta- and para-sirtinol, 7 and 8) have also been prepared. In Example 10, we report the synthesis of enantiomerically pure (R)- and (S)-sirtinol (compounds 9 and 10) (FIG. 1). Compounds 2-10 have been tested as inhibitors of yeast Sir2 and human SIRT1 in vitro, with 1 as a reference compound. Moreover, phenotypic screening involving SIR2-mediated URA3 gene silencing (FIG. 2) has been performed on meta-, para-, (R)-, and (S)-sirtinol derivatives 7-10, in order to evaluate their ability to function in vivo. By molecular modeling and inhibitor docking, we investigated the mechanism of inhibition by meta- and para-sirtinol 7 and 8.

Condensation between 1-naphtaldehyde and 2-amino-N-(1-phenylethyl)benzamide^{48,49} in acidic medium afforded the 2-[(1-naphthalenylmethylene)amino]-N-(1-phenylethyl)benzamide 2, that is the 2-dihydroxynaphthyl analogue of sirtinol 1 (Scheme 1). By heating 2-hydroxy-1-naphtaldehyde with the appropriate aniline derivatives (ethyl 2-aminobenzoate, 2-aminobenzoic acid, 2-aminobenzamide, 2-aminobenzonitrile) in acidic medium, the desired sirtinol analogues (3-6 have been obtained in high yields (Scheme 1).

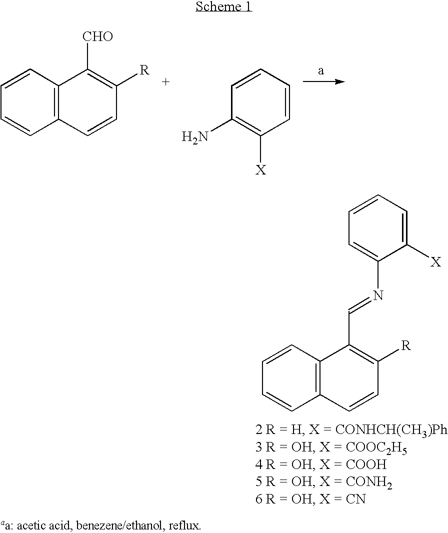

Finally, the known 3- and 4-amino-N-(1-phenylethyl)benzamides 11⁴⁹ and 12⁴⁹ have been condensed with 2-hydroxy-1-naphtaldehyde in acidic medium gave the two sirtinol isomers 3- and 4-[(2-hydroxy-1-naphthalenylmethylene)amino]-N-(1-phenylethyl)benzamide 7 and 8 (meta-sirtinol and para-sirtinol, respectively) (Scheme 2).

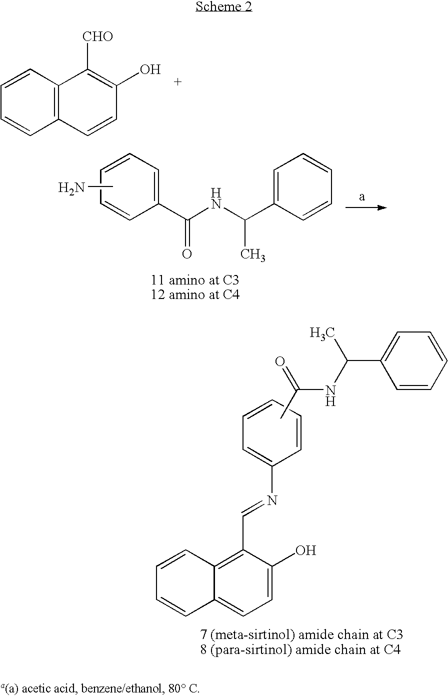

Chemical and physical data of compounds 2-8 are listed in Table 1.

TABLE 1
Chemical and Physical Data for Compounds 2-8

| compd | mp ° C. | recrystn solvent | % yield | formula | anal^{a} |
|---|---|---|---|---|---|
| 2 | 101-103 | methanol | 74 | C₂₆H₂₂N₂O | C, H, N |
| 3 | oil | | 54 | C₂₀H₁₇NO₃ | C, H, N |
| 4 | 180-182 | acetonitrile | 87 | C₁₈H₁₃NO₃ | C, H, N |
| 5 | 238-240 | acetonitrile | 91 | C₁₈H₁₄N₂O₂ | C, H, N |
| 6 | 139-141 | acetonitrile | 94 | C₁₈H₁₂N₂O | C, H, N |
| 7 | 125-126 | acetonitrile | 92 | C₂₆H₂₂N₂O₂ | C, H, N |
| 8 | 128-130 | acetonitrile | 90 | C₂₆H₂₂N₂O₂ | C, H, N |

^{a}Analytical results were within ±0.4% of the theoretical values.

The sirtinol analogues differ in the functional group at the benzene 2′ position and the degree of inhibition was assessed in vitro using recombinant yeast Sir2 and human SIRT1, and in a yeast phenotypic assay. Two analogues, namely 3- and 4-[(2-hydroxy-1-naphthalenylmethylene)amino]-N-(1-phenylethyl)benzamide (ie, meta- and para-sirtinol) were 2- and 8-fold more potent than sirtinol, respectively, against human SIRT1. These two compounds were more potent Sir2 inhibitory activity than sirtinol in the yeast in vivo assay. Compounds lacking the 2-hydroxy group at the naphthalene moiety or bearing several modifications at the benzene 2′-position of the aniline portion (carbethoxy, carboxy, and cyano), were 1.3-13 times less potent than sirtinol, whereas a 2′-carboxamido analogue was totally inactive. Both (R)- and (S)-sirtinol had similar inhibitory effects on the yeast and human enzymes demonstrating no enantioselective inhibitory effect.

A series of sirtinol analogues 2-8, R-(9) and S-sirtinol (10) have been designed and synthesized and their inhibitory action together with that of sirtinol (1) have been tested against yeast Sir2 and human SIRT1 enzymes (See Example 3). Phenotypic screening based on inhibition of Sir2-mediated telomeric URA3 gene silencing has been performed on 1 and 7-10 (See Example 4). Enzyme inhibitory data showed that compounds 2-4,6, lacking the 2-hydroxy group at the naphthalene moiety (2) or bearing several modifications at the benzene 2′-position of the aniline portion (carbethoxy (3), carboxy (4), and cyano (6)) of sirtinol 1, were 3-13 (or 1.3-5) times less potent than 1 against yeast Sir2 (or human SIRT1), while the 2′-carboxamido analogue 5 was totally inactive in both enzyme assays. 3- And 4-[(2-hydroxy-1-naphthalenylmethylene)amino]-N-(1-phenylethyl)benzamide 7 and 8 (meta- and para-sirtinol) showed, in ySir2 inhibiting assay, IC₅₀ values similar to that of 1, whilst against hSIRT1 they were 2.2- (7) and 10-fold (8) more potent than sirtinol 1. Furthermore, in phenotypic screening 7 and 8 were more potent inhibitors than 1. Interestingly, in the 7 and 8 structures (meta- and para-sirtinol) the formation of the hydrogen bond observed in sirtinol 1 between the naphthyl 2-hydroxy group, the aldimine nitrogen, and the carbonyl of the amide function⁵⁴ is energetically unfavorable.

Against ySir2, (R)-(9) and (S)-sirtinol (10) were similar in potency compared to 1, whereas against hSIRT1, 9 and 10 were 2-fold more potent. In either case, there was no enantioselective inhibitory effect. Docking studies described in Example 10 reveal that (R)- and (S)-sirtinol (9 and 10) might act as Ac—K binding site competitors and block the entrance channel of the acetylated lysine by mimicking the backbone of the peptide substrate. Using molecular modeling to dock both meta- and para-sirtinol to a known Sir2 structure, we provide evidence that these compounds bind the enzyme more efficient either at the acetyl-lysine (Ac—K) or NAD⁺ binding sites, thus accounting for their increased potency relative to sirtinol. Unlike 9 and 10, meta- and para-sirtinol (7 and 8) are predicted to bind the Sir2-Af2 enzyme in a more efficient fashion by fully occupying the Ac—K or the NAD⁺ substrate binding site. Further computational and synthetic studies are in progress to design new sirtinol-related Sir2 and Sir2-like inhibitors.

### Example 2

### Materials and Methods for Examples 3-9

### Chemistry

Melting points were determined on a Bütchi 530 melting point apparatus and are uncorrected. Infrared (IR) spectra (KBr) were recorded on a Perkin-Elmer Spectrum One instrument. ¹H NMR spectra were recorded at 200 MHz on a Bruker AC 200 spectrometer; chemical shifts are reported in δ (ppm) units relative to the internal reference tetramethylsilane (Me₄Si). All compounds were routinely checked by TLC and ¹H NMR. TLC was performed on aluminum-backed silica gel plates (Merck DC-Alufolien Kieselgel 60 F₂₅₄) with spots visualized by UV light. All solvents were reagent grade and, when necessary, were purified and dried by standards methods. Concentration of solutions after reactions and extractions involved the use of a rotary evaporator operating at a reduced pressure of ca. 20 Torr. Organic solutions were dried over anhydrous sodium sulfate. Analytical results are within ±0.40% of the theoretical values. All chemicals were purchased from Aldrich Chimica, Milan (Italy) or Lancaster Synthesis GmbH, Milan (Italy) and were of the highest purity.

### Syntheses

The specific examples presented below illustrate general synthetic methods. As a rule, samples prepared for physical and biological studies were dried in high vacuum over P₂O₅ for 20 h at temperatures ranging from 25 to 110° C., depending on the sample melting point.

### General Procedure for the Synthesis of Sirtinol Analogues 2-8

The synthesis of 4-[(2-hydroxy-1-naphthalenylmethylene)amino]-N-(1-phenylethyl)benzamide (8) is provided as an exemplary synthesis of a sirtinol analogue. A mixture of 2-hydroxy-1-naphtaldehyde (0.5 g, 2.1 mmol) and 4-amino-N-(1-phenylethyl)benzamide 12⁴⁹ (0.4 g, 2.1 mmol) in 30 mL of absolute ethanol:benzene (2:1) in the presence of a catalytic amount of glacial acetic acid was heated at reflux for 4 h. After cooling at room temperature, from the mixture reaction a yellow solid was formed, which was collected by filtration, washed with CHCl₃ and purified by crystallization. ¹H NMR (DMSO-d₆) □ 1.47-1.48 (d, 3H, CHCH₃), 5.16-5.19 (q, 1H, CHCH₃), 6.94-6.96 (d, 1H, NH), 7.20-7.21 (m, 1H, naphthyl H-3), 7.29-7.54 (m, 7H, Ph and benzene H-3′,5′), 7.69-7.76 (m, 2H, naphthyl H-6,7), 7.89-8.01 (m, 3H, naphthyl H-8 and benzene H-2′,6′), 8.46-8.48 (m, 1H, naphthyl H-5), 8.83-8.85 (m, 1H, naphthyl H-4), 9.64 (s, 1H, CH═N), 14.25 (s, 1H, OH). Anal. (C₂₆H₂₂N₂O₂) C, H, N.

### Enzymatic Assays

Recombinant His-tagged yeast ySir2 and His-tagged hSIRT1 were purified and assayed for deacetylase activity using the HDAC fluorescent activity assay/drug discovery kit (AK-500, BIOMOL Research Laboratories).⁵⁵ This assay system allows detection of a fluorescent signal upon deacetylation of a histone substrate when treated with developer. Fluorescence was measured on a fluorometric reader (Wallac Victor III fluorescence plate reader Perkin Elmer) with excitation set at 360 nm and emission detection set at 450 nm. Reactions consisted of either 3 μg of ySir2 or 1 μg of SIRT1, incubated with 250 μM acetylated histone substrate, 1 mM dithiothreitol, and a range of inhibitor concentrations as described. Reactions with the yeast and human proteins were carried out at 30 and 37° C., respectively, for 60 min. Assays were performed in the presence of 200 μM NAD⁺ and each of the inhibitors at 0, 20, 75, 100, 150, or 300 μM concentrations.

### Telomeric Silencing Assay

Wildtype (AYH2.45)⁵⁰ and sir2Δ (STY30)⁵⁰ strains were grown to exponential phase (0.5 OD/mL) in YPD medium and cells were spotted in 10-fold dilutions on SC medium containing 1 mg/mL 5-fluoroorotic acid (5-FOA).⁵¹ When appropriate, cells were grown on plates containing 0.18% DMSO and the specified concentration of Sir2 inhibitor (5.6 and 16.7 μM). Cell growth was evaluated after 3-4 d at 30° C.

### Molecular Modeling and Docking Studies

All molecular modeling calculations and manipulations were performed using the software packages Macromodel 7.1,⁵⁶ MOPAC 2000,^{57,58} Deep View Swiss-PdbViewer 3.7,⁵⁹ ADT 1.3,⁶⁰ and Autodock 3.0.5^{61,62} running on IBM compatible AMD Athlon workstations. Images were generated using Chimera 1.2056⁶³ was used. For the conformational analysis and for energy minimization, the all-atom Amber force field⁶⁴ was generated using the Macromodel package.

### Meta- and Para-Sirtinol (7 and 8): Structure Preparation and Docking

The structures of (R)-meta-sirtinol ((R)-7), (S)-meta-sirtinol ((S)-7), (R)-para-sirtinol ((R)-8), and (S)-para-sirtinol ((S)-8) were drawn by the Macromodel graphical interface Maestro 3.0 and geometry optimized using a molecular dynamic protocol similar to that used in the optimization of the Sir2-Af2/p53 complex.⁴⁸ Each molecule was energy-minimized to a low gradient. The non-bonded cut-off distances were set to 20 Å for both Van der Waals and electrostatic interactions. An initial random velocity to all atoms corresponding to 300 K was applied. Three subsequent molecular dynamics runs were then performed. The first was carried out for 10 ps with a 1.5 fs time step at a constant temperature of 300 K for equilibration purposes. The next molecular dynamic was carried out for 20 ps, during which the system is coupled to a 150 K thermal bath with a time constant of 5 ps. The time constant represents approximately the half-life for equilibration with the bath. Consequently the second molecular dynamic command caused the molecule to slowly cool to approximately 150 K. The third and last dynamic cooled the molecule to 50 K over 20 ps. A final energy minimization was then carried out for 250 iterations using a conjugate gradient. The minimizations and the molecular dynamics were in all cases performed using the continuum solvent simulation (GBSA keyword). The binding modes of the Sir2 small molecule inhibitors were analyzed by a docking procedure using the program Autodock. For the docking, a grid spacing of 0.375 Å and 70×72×72 number of points were used. The grid was centered on the mass center of the experimental p53 Ac—K coordinates, and comprised more than 5 Å of both the NAD⁺ and Ac—K binding sites. The GA-LS method was adopted using the default settings. Amber united atoms for the protein and Gasteiger atom charge were for the inhibitors were assigned by the means of the program ADT. Autodock generated 100 possible binding conformations clustered in 2.0 Å. Three docking experiments were carried out for each inhibitor using the Sir2-Af2 enzyme in different compositions: (i) the free sir2-Af2 enzyme, (ii) the Sir2-Af2/NAD⁺ and (iii) Sir2-Af2/p53 binary complexes.

### Example 3

### Yeast Sir2 and Human SIRT1 Inhibitory Assays

The novel sirtinol analogues 2-8, together with sirtinol (1) and its enantiomers (R)-(9) and (s)-sirtinol (10), have been evaluated for their ability to inhibit yeast Sir2 (ySir2) and human SIRT1 (hSIRT1) enzymes. The results expressed as the percent of inhibition at 100 μM and IC₅₀ (50% inhibitory concentration) values are reported in Table 2.

TABLE 2
Yeast Sir2 (ySir2) and Human SIRT1 (hSIRT1) Inhibitory Activity of Sirtinol Analogues 1-10^{a}

| | | % inhibtn | | IC₅₀ | |
|---|---|---|---|---|---|
| | | (100 μM) | | (μM) | |
| compd | structure | ySir2 | hSIRT1 | ySir2 | hSIRT1 |
| 1 (sirtinol) | 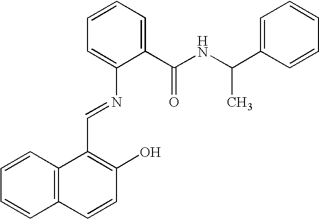 | 65.6^{b} | 44.5 | 48 ± 4^{b} | 131 ± 11 |
| 2 | 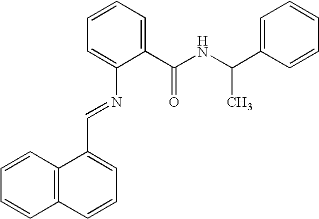 | 10.0 | 33.0 | ND^{c} | ND |
| 3 | 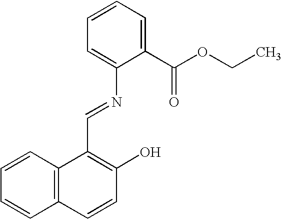 | 20.0 | 9.0 | ND | ND |
| 4 | 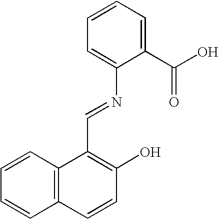 | 5.0 | 26.0 | ND | ND |
| 5 | 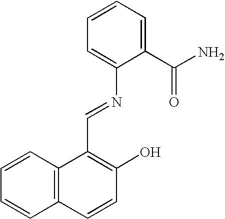 | NI^{d} | NI | | |
| 6 | 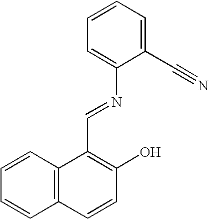 | 15.0 | 20.0 | ND | ND |
| 7 (meta-sirtinol) | 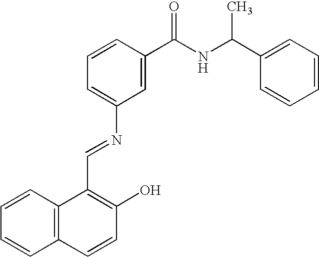 | 53.9 | 63.0 | 72 ± 3 | 59 ± 2 |
| 8 (para-sirtinol) | 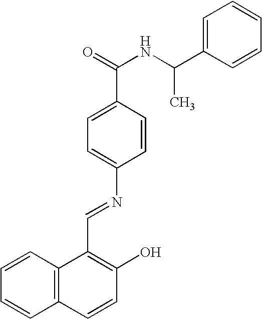 | 59.8 | 82.6 | 33 ± 1 | 13 ± 2 |
| 9 ((R)-sirtinol) | 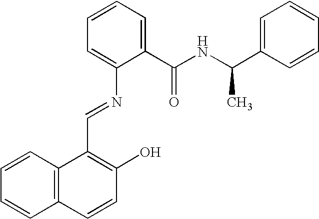 | 55.5^{b} | 61.8 | 62 ± 5^{b} | 55 ± 5 |
| 10 ((S)-sirtinol) | 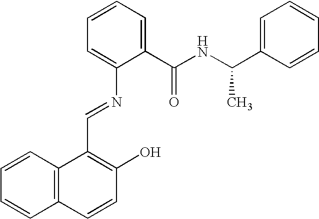 | 57.0^{b} | 60.2 | 66 ± 4^{b} | 67 ± 4 |

^{a}Data represent mean values of at least three separate experiments.
^{b}Reference 48.
^{c}ND, not determined.
^{d}NI, no inhibition.

When tested against ySir2, sirtinol (1) showed 65.6% of inhibition at 100 μM, its IC₅₀ being 48 μM (Table 2). Deletion of the hydroxyl group at C2 position of the naphthalene moiety furnished a compound (2) that was 7-fold less potent than sirtinol in inhibiting ySir2, thus confirming the important role of the 2-hydroxy-1-naphtaldehyde moiety in inhibiting ySir2, as previously reported by Grozinger et al. Replacement of the N-1-phenylethylamide moiety of sirtinol with a carbethoxy, carboxy, or cyano group lowered the Sir2 inhibitory activity of the derivatives (3, 3-fold; 4, 13-fold; 6, 4-times), and the insertion at the aniline C2 position of a carboxyamide portion resulted in a total loss of Sir2 inhibitory activity (compound 5). When the N-1-phenylethylamide moiety was shifted from the ortho position to the meta or para position of the benzene ring (compounds 7 and 8), a slightly less (1.5-fold, 7) or more potent (1.5-fold, 8) compound than 1 was obtained, respectively. (R)- and (S)-sirtinol (9 and 10), showed IC₅₀ values similar to that of sirtinol 1 (Table 2), thus demonstrating the lack of enantioselectivity in sirtinol inhibitory activity.

Compared to its ability to inhibit ySir2,⁴² sirtinol (1) was a weak inhibitor of hSIRT1, with 44.5% of inhibition at 100 μM and IC₅₀=131 μM (Table 1). 2-Dehydroxy analogue 2 as well as 2′-carbethoxy- (3), 2′-carboxy- (4), and 2′-cyano- (6) derivatives were less potent than 1 in inhibiting hSIRT1 (1.3 to 5 fold). As for ySir2, the 2′-carboxyamide 5 was totally inactive against hSIRT1. In contrast, 3-[(2-hydroxy-1-naphthalenylmethylene)amino]-N-(1-phenylethyl)benzamide (meta-sirtinol) 7 was 2.2-fold more potent than sirtinol in inhibiting hSIRT1, and its para-isomer 8 inhibited hSIRT1 10 times more efficiently than 1, with IC₅₀=13 μM. Against the yeast enzyme, the IC₅₀ values of (R)- and (S-)sirtinol (9 and 10) are 55 and 67 μM, respectively, demonstrating that there is no difference of activity between the two enantiomers and sirtinol (1), but they were 2-fold more potent than 1 in inhibiting human SIRT1 (Table 1).

### Example 4

### Phenotypic Screening: Telomeric Sir2-Mediated URA3 Gene Silencing

Compounds 1 and 7-10 were evaluated for in vivo activity using a functional test for telomeric silencing in yeast. The yeast strain contained a URA3 reporter gene integrated into the sub-telomeric region of chromosome VII-L where it is silenced by Sir2.⁵⁰ In the presence of 5-fluoroorotic acid (5-FOA), loss of transcriptional silencing in this strain and the corresponding increase in URA3 expression is lethal (FIG. 2).⁵¹

Growth of the URA3-tagged strain was dramatically reduced relative to the untreated cells (upper panel, a sir2Δ strain was used as positive control for complete loss or silencing of Sir2 activity), in a dose-dependent manner by the addition of 5.6 μM, 16.7 μM and 25 μM of the Sir2 inhibitors 1 and 7-10. No cytotoxic effect was observed for these inhibitors at 16.7 and 25 μM. A semi-quantitative assay of the spots on the plates treated with 5-FOA and the Sir2 inhibitors at 16.7 μM concentration was performed by densitometric scanning of the plates. By this method, meta- and para-sirtinol (7 and 8) were more potent than sirtinol, while the relative potencies of the two sirtinol enantiomers (R) 9 and (S) 10 were similar to each other and to their racemate, thus confirming the lack of enantioselective inhibitory action of these derivatives.

### Example 5

### Binding Mode Analysis of Formulas 7 and 8

Docking studies using the Autodock software suite^{50,51} were undertaken on derivatives 7 and 8 to explore the mechanism by which these molecules inhibit sirtuins. The free Sir2-Af2 enzyme and the binary complexes with p53 substrate obtained from a modeled Sir2-Af2/NAD⁺/p53 ternary complex⁴⁶ were used for docking studies. Because 7 and 8 contain a chiral center, the binding mode of both their (R) and (S) enantiomers were explored. The Sir2-Af2/NAD⁺ complex was not taken into consideration in these experiments for two reasons. Firstly, Borra et al. reported that NAD⁺ would likely bind only to the pre-formed Sir2/acetylated peptide binary complex.²⁴ Secondly, previous docking studies with the Sir2-Af2/NAD⁺ complex⁴⁶ confirmed that the inhibitors would only bind reasonably to the free and p53 bound enzyme.

### Example 6

### Docking of (R)-meta-sirtinol ((R)-7)

Based on the structure of the free Sir2-Af2 enzyme, (R)-7 was found to dock in the Ac—K binding site, partially overlapping also the NAD⁺ volume, where it could act as a competitive inhibitor for both the acetylated substrate and NAD⁺. When docked to the Sir2-Af2/p53 complex, (R)-7 binds preferably in the NAD⁺ binding site suggesting the R enantiomer of 7 could act as a competitive inhibitor of Sir2-Af2 by inhibiting the formation of the Sir2-Af2/p53 complex.

### Example 7

### Docking of (S)-meta-sirtinol ((S)-7)

Although the R enantiomer of meta-sirtinol (7) might bind in the Ac—K binding site, Autodock simulations using either the free or the p53-bound enzyme indicate that the S counterpart ((S)-7) might preferentially compete with NAD⁺. In this NAD⁺ competitive conformation, (S)-7 makes numerous positive interactions: a hydrogen bond between the carboxamide oxygen and the hydroxylic of Ser193 (distance C═O_{(S)-7}—γO_{Ser193}=2.87 Å), a hydrogen bond between the naphtholic hydroxyl and the carbonyl oxygen of the acetyl group of Ac—K (distance naphth-O_{(S)-7}—O═C_{Ac—K}=2.18 Å), positive hydrophobic interactions of the naphthyl ring in a hydrophobic pocket formed by the side chains of Phe35, Ala51, Phe63, Phe66 and Ile102 (not shown). Note that in this model, the sirtinol volume overlaps the NAD⁺ volume.

### Example 8

### Docking of (R)-para-sirtinol ((R)-8)

Similar to (S)-meta-sirtinol ((S)-7), the docked conformations of (R)-8 overlap the NAD⁺ binding site, although there is less overlap with NAD⁺. Based on the free Sir2-Af2 model, (R)-8 forms a hydrogen bond between the carboxamide oxygen and the side chain amide group of Gln100 (distance C═O_{(R)-8}—N—C═O_{Gln100}=2.74 Å), while in the Sir2-Af2/p53 model, (R)-8 seems to make two hydrogen bonds, one between the carboxamide oxygen and the NAD⁺ anchoring side chain of Arg36 (distance C═O_{(R)-8}—N_{Arg36}=2.88 Å) and the other between the (R)-8 amide NH and the C═O of Gly191 (distance CO—N_{(R)-8}—O═C_{Gly191}=2.67 Å).

### Example 9

### Docking of (S)-para-sirtinol ((S)-8)

Unlike the R stereoisomer, (S)-8 did not show a unique binding mode. The free enzyme docked conformation, similarly to that of (R)-7, binds fulfilling the Ac—K site and partially covering the NAD⁺ nicotinamide binding pocket. On the other hand, the docking in the p53 bound Sir2-Af2 complex suggest (S)-8 to bind exclusively in the NAD⁺ site. No particular interaction can be observed for the two docked conformations.

### REFERENCES FOR EXAMPLES 1-9

(1) Kurdistani et al. Nat. Rev. Mol. Cell Biol. 2003, 4, 276-284; (2) Wu et al. Trends Biochem. Sci. 2000, 25, 619-623; (3) Urnov et al. Emerg. Ther. Targets 2000, 4, 665-685; (4) Pazin et al. Cell 1997, 89, 325-328; (5) Grunstein et al. Nature 1997, 389, 349-352; (6) Davie et al. Curr. Opin. Genet. Dev. 1998, 8, 173-178; (7) Kouzarides et al. Genet. Dev. 1999, 9, 40-48; (8) Archer et al. Opin. Genet. Dev. 1999, 9, 171-174; (9) Cress et al. J. Cell. Physiol. 2000, 27, 268-271; (10) Grozinger et al. Chem. Biol. 2002, 9, 3-16; (11) Mai et al. Med. Res. Rev. 2005, in press; (12) Lusser et al. Science 1997, 277, 88-91; (13) Miller et al. J. Med. Chem. 2003, 46, 5097-5116; (14) Fumurai et al. Proc. Natl. Acad. Sci. USA 2001, 98, 87-92; (15) Kruh et al. Mol. Cell. Biochem. 1982, 42, 65-82; (16) Fumurai et al. Cancer Res. 2002, 62, 4916-4921; (17) Stemson et al. Org. Lett. 2001, 3, 4239-4242; (18) Wong et al. J. Am. Chem. Soc. 2003, 125, 5586-5587; (19) Haggarty et al. Chem. Biol. 2003, 10, 383-396; (20) Haggarty et al. Proc. Natl. Acad. Sci. USA 2003, 100, 4389-4394; (21) Ma et al. J. Med. Chem. 2003, 46, 4826-4829; (22) Sauve et al. Biochemistry 2003, 42, 9249-9256; (23) Tanny et al. Cell 1999, 99, 735-745; (24) Tanner et al. Proc. Natl. Acad. Sci. USA 2000, 97, 14178-14182; (25) Chang et al. J. Biol. Chem. 2002, 277, 34489-34498; (26) Borra et al. Biochemistry 2004, 43, 9877-9887; (27) Lamming et al. Mol. Microbiol. 2004, 53, 1003-1009; (28) Blander et al. Annu. Rev. Biochem. 2004, 73, 417-435; (29) Muller et al. Mech. Ageing Dev. 1980, 12, 47-52; (30) Lakowski et al. Proc. Natl. Acad. Sci. USA 1998, 95, 13091-13096 (31) Weindruch et al. J. Nutr. 1986, 116, 641-654; (32) Roth et al. J. Am. Geriatr. Soc. 1999, 47, 896-903; (33) Vaziri et al. Cell, 2001, 107, 149-159; (34) Avalos et al. Mol. Cell 2002, 10, 523-535; (35) Muth et al. EMBO J. 2001, 20, 1353-1362; (36) North et al. Mol. Cell 2003, 11, 437-444; (37) Bell et al. Science 2002, 296, 148-151; (38) Starai et al. Science 2002, 298, 2390-2392; (39) Starai et al. Curr. Opin. Microbiol. 2004, 7, 115-119; (40) Borra et al. J. Biol. Chem. 2002, 277, 12632-12641; (41) Kyrylenko et al. Cell. Mol. Life Sci. 2003, 60, 1990-1997; (42) Grozinger et al. J. Biol. Chem. 2001, 276, 38837-38843; (43) Bedalov et al. Proc. Natl. Acad. Sci. USA 2001, 98, 15113-15118; (44) Bitterman et al. J. Biol. Chem. 2002, 47, 45099-45107; (45) Hirao et al. J. Biol. Chem. 2003, 278, 52773-52782; (46) Posakony et al. J. Med. Chem. 2004, 47, 2635-2644; (47) Jackson et al. J. Biol. Chem. 2003, 278, 50985-50998; (48) Mai et al. J. Med. Chem. submitted; (49) Clark et al. J. Med. Chem. 1987, 30, 1214-1218; (50) Hecht et al. Nature 1996, 383, 92-96; (51) Boeke et al. Methods Enzymol. 1987, 154, 164-175; (52) Goodsell et al. J. Mol. Recognit. 1996, 9, 1-5; (53) Oshiro et al. J. Comput.-Aided Mol. Des. 1995, 9, 113-130; (54) Bernstein et al. Chem. & Biol. 2002, 9, 1167-1173; (55) Howitz et al. Nature 2003, 425, 191-196; (56) Mohamadi et al. J. Comput. Chem. 1990, 11, 440-467; (57) Stewart et al. J. Comput.-Aided Mol. Des. 1990, 4, 1-105; (58) MOPAC 2000.00 Manual; Stewart, J. J. P., Ed; Fujitsu Limited: Tokyo, Japan 1999; (59) Guex et al. Electrophoresis 1997, 18, 2714-2723. (See, e.g., the world wide web at extension expasy.org/spdvy); (60) Sanner et al. Proc. Pacific Symposium in Biocomputing 1998, pp 401-412; (61) Goodsell et al. J. Mol Recognit. 1996, 9, 1-5; (62) Oshiro et al. J. Comput.-Aided Mol. Des. 1995, 9, 113-130; (63) Pettersen et al. J. Comput. Chem. 2004, 25, 1605-1612. (See, e.g., the world wide web at extension cgl.ucsf.edu/chimera); (64) Pearlman et al. Comput. Phys. Commun. 1995, 91, 1-41.

### Example 10

### Molecular Modeling Studies

The structures of four sirtuins, namely Archaeoglobus fulgidus Sir2-Af ^{16,26} and Sir2-Af2,²⁷ human SIRT2,²⁸ and yeast Hst2²⁹, have been obtained at atomic resolution and a number of common features can be drawn. Overall, Sir2 enzymes possess a large, conserved core containing a Rossmann fold that binds NAD⁺ and a smaller domain that forms a Zn-binding domain and a helical module.

Although structures of various sirtuins have been solved, there have been no detailed modeling studies on the known synthetic small-molecule sirtuin inhibitors, namely A3,³⁰ M15,³⁰ splitomicin³¹ and sirtinol 1³⁰ (Chart 1).

Chart 1: Structures of small molecule Sir2 inhibitors.

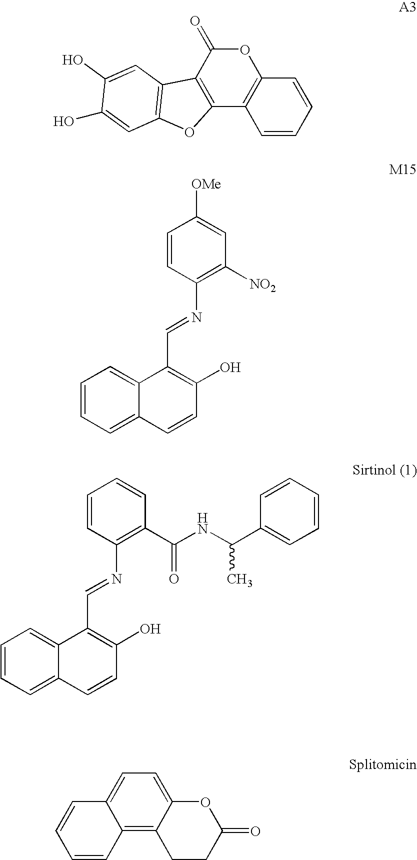

As described herein, we investigated the binding of small-molecule sirtuin inhibitors using docking studies performed on the X-ray structure of Archaeoglobus fulgidus Sir2-Af2. To complement this analysis, we have synthesized the two (R)- and (S)-sirtinol enantiomers (compounds 9 and 10, described above and chart 2), and compared these as inhibitors of yeast Sir2 to the racemic form sirtinol 1.

Chart 2: Structures of sirtinol enantiomers (R)-sirtinol (9) and (S)-sirtinol (10).

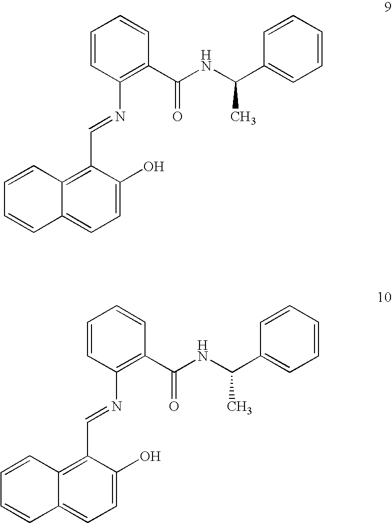

### Molecular Modelling Methods

Sir2-Af2/NAD⁺/p53 Complex Structure Preparation. The thermophile Archaeoglobus fulgidus encodes two Sir2 homologues, Sir2-Af1 and Sir2-Af2, which consist of minimal conserved enzymatic core and share 46% amino acid sequence identity. In vitro, Sir2-Af2 has been shown to deacetylate peptides corresponding to the C-terminus of p53³² exhibiting distinctly higher rates of enzymatic activity on this substrate than the Sir2-Af1. Moreover, Sir2-Af1 has been reported to deacetylate the acetyl group from acetylated bovine serum albumin (BSA), whereas Sir2-Af2 removes the acetylated N-terminal tails of histones.^{26,33} Because Sir2-Af2 has been well characterized with a natural substrate, we decided to use this enzyme as a starting point for docking simulations (coordinates obtained from Brookhaven Protein Database³⁴, PDB entry code 1ma3).

The deposited structure of PDB 1ma3 lacked two important structural features: the NAD⁺ substrate and a small portion of the flexible loop region (residues 29-48)¹⁶ in the helical region.²⁷ Both the NAD⁺ and the loop region are important for the enzymatic mechanism of the Sir2 proteins.¹⁶ Therefore a Sir2-Af2/NAD⁺/p53 ternary complex was first constructed using structural information contained in the recently reported Sir2-Af1/NAD⁺ complexes (PDB entry codes 1m2g¹⁶ and 1ici²⁶). Firstly, the ten missing residues of the loop region (residues 30-39) were inserted and the overall Sir2-Af2/p53 complex was submitted to a molecular dynamic run with the Macromodel software to optimize the structure. Secondly, the bound conformation of the NAD⁺ substrate was investigated by means of an automatic docking procedure with the program Autodock. Finally, the results of the docking were rescored by a single point energy minimization of the ternary complexes. The global minimum of the minimized complexes was then used for subsequent docking studies on the known Sir2 inhibitors A3,³⁰ M15,³⁰ splitomicin,³¹ and the two sirtinol enantiomers 9 and 10. While this paper was in preparation, five new structures of Sir2-Af2 were released in the PDB:³⁵ although some structural features are of great interest, none of them gave additional information on the Sir2-Af2/NAD⁺/peptide ternary complex.

Docking of the Sir2 inhibitors. Molecular docking of A3,³⁰ M15,³⁰ splitomicin,³¹ and the two sirtinol enantiomers 9 and 10 were performed on the above Sir2-Af2 structure using the Autodock program. No experimental information are available whether or not the five inhibitors act as competitive agonist of the NAD⁺ or the Ac—K (Aly) substrates, and if they are non-competitive inhibitors similarly to nicotinamide.³⁶ To address the putative mechanism of action, the docking experiments were conducted as following: 1) the NAD⁺ and the p53 substrates were removed either in turn or together from the ternary complex; 2) the five inhibitors were docked in turn i) in the NAD⁺ site using the binary complex Sir2-Af2/p53, ii) in the p53 binding site using the Sir2-Af2/NAD⁺ binary complex, and iii) in both the Ac—K and NAD⁺ binding sites using the free Sir2-Af2 enzyme deprived of the two substrates. Since the macromolecule structure was optimized as a ternary complex (productive model of the complex),³⁵ no investigations were undertaken in a possible allosteric inhibition mechanism. In parallel experiments either a capped Ac—K residue or p53 found in the PDB 1ma3 complex were docked back to the Sir-Af2 for docking assessment.

### Chemistry

Catalytic reduction of known (R/S)-, (R)-, and (S)-2-nitro-N-(1-phenylethyl)benzamides 20-22^{37,38} prepared by standard methods afforded the corresponding (R/S)-,³⁷ (R)-, and (S)-2-aminobenzamides 23-25, which were in turn condensed with 2-hydroxy-1-naphtaldehyde in acidic medium to give sirtinol 1 and (R)- and (S)-sirtinol, 9 and 10 respectively (Scheme 3).

Chemical and physical data of compounds 1, 9, 10, 24 and 25 are listed in Table 3. Scheme 3^{a}

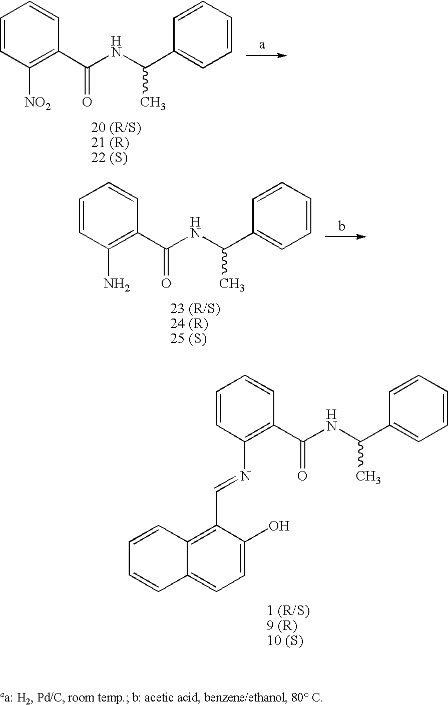

TABLE 3
Chemical and Physical Data for Compounds 1, 9, 10, 24 and 25

| | | | | % | | |
|---|---|---|---|---|---|---|
| compd | mp ° C. | [α]_{D} | recrystn solvent | yield | formula | anal^{a} |
| 1 | 119-120 | — | acetonitrile | 94 | C₂₆H₂₂N₂O₂ | C, H, N |
| 9 | 123-124 | +3.3^{b} | acetonitrile | 93 | C₂₆H₂₂N₂O₂ | C, H, N |
| 10 | 117-119 | −3.9^{b} | acetonitrile | 94 | C₂₆H₂₂N₂O₂ | C, H, N |
| 24 | 157-159 | +1.1^{c} | CH₂Cl₂/n- | 91 | C₁₅H₁₆N₂O | C, H, N |
| | | | hexane | | | |
| 25 | 153-154 | −1.0^{c} | CH₂Cl₂/n- | 93 | C₁₅H₁₆N₂O | C, H, N |
| | | | hexane | | | |

^{a}Analytical results were within ±0.4% of the theoretical values.
^{b}Concentration in MeOH: 0.001 g/mL.
^{c}Concentration in CHCl₃: 0.016 g/mL.

### Results and Discussion

Molecular Modeling. Sir2-Af2/NAD⁺/p53 Complex. The structure of Sir2-Af2/NAD⁺/p53 ternary complex was processed by molecular dynamics (MD) simulation as reported in Experimental Methods. The Sir2-Af2/NAD⁺/p53 optimized structure compared to the Sir2-Af2/p53 experimental structure (PDB entry code 1ma3) showed an all-heavy-atoms root mean square distance (RMSD) of only 1.26. This value suggested that the quality of the final Sir2-Af2/NAD⁺/p53 ternary complex structure was very high and suitable for binding site analysis. While this paper was in preparation Avalos et al. reported the Sir2-Af2/NAD⁺ complex in different conformations (PDB entry code 1s7g);³⁵ they found that the NAD⁺ co-substrate can be observed in productive and non-productive binding conformations suggesting a new structure-based mechanism of action. A comparison of the modelled flexible β1-α2 loop (residues 30-39)¹⁶ in the ternary complex with that reported in the PDB 1s7g complex³⁵ showed an RMSD value of 1.249, which further supports the goodness of our calculations.

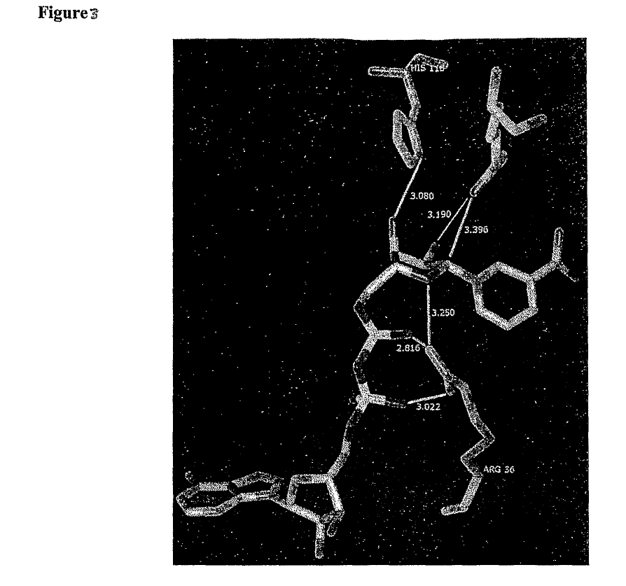

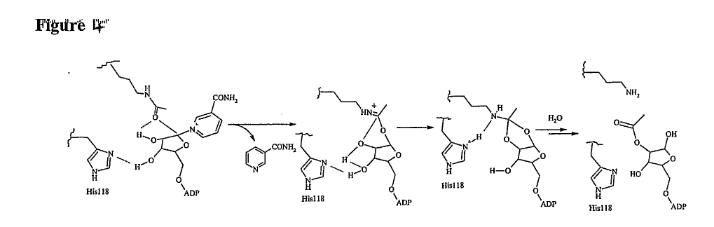

The structure of the Sir2-Af2/NAD⁺/p53 ternary complex as described herein suggests an enzymatic mechanism of action that is somehow in good agreement with both the Zhao et al.³⁹ and the Avalos et al.³⁵ recently reported revised enzymatic mechanisms. The ternary complex NAD⁺ bound structure compared to the previously observed binding conformations in either the Sir2-Af1 (PDB entry code 1ici)²⁶ or the Sir2-Af2 (PDB entry code 1s7g)³⁵ shows a good superimposition of the adenine ribose moiety in which the phosphate bridge makes two hydrogen bonds with the Arg36 (FIG. 3). On the other hand, the nicotinamide ribose extremity is placed in half way between the non-productive and the productive conformations described by Avalos et al. In the ternary complex the NAD⁺ seems to make a hydrogen bond between the nicotinamide ribose 3′-OH and the His118 (distance 3.080 Å), at the same time the Ac—K carbonyl oxygen is both at hydrogen bond distance to the nicotinamide ribose 2′-OH (distance CO_{Aly}—3′-O_{ribose}=3.190 Å) and at reaction distance to the nicotinamide positively charged nitrogen (distance CO_{Aly}—-N⁺_{nicotinamide}=3.396 Å). This scenario suggests that the initial reaction, that leads to the nicotinamide moiety cleavage transition state, is likely due to a complicate net of interactions in which the action of His118 seems to be extremely important. The crucial role of the His118 is threefold, firstly it contributes to anchor the NAD⁺ (hydrogen bond to 3′-OH) allowing the approaching of NAD⁺ to the Ac—K residue (hydrogen bond acetyllysine-NAD⁺), secondly it activates the 2′-OH through 3′-OH deprotonation, leading to the internal attack to the Ac—K carbon atom and finally the protonated His118 releases a hydrogen atom to the lysine ε-amino group leading to the final amide hydrolysis and the release of the deacetylated peptide and 2′-O-acetyl-ADP-ribose, likely mediated by a water molecule (FIG. 4).

Since the NAD⁺ binding mode was calculated in the presence of the acetylated p53, we believe that this could reflect a realistic view of the ongoing NAD⁺ cleavage and acetyl transferring from the acetylated peptide to the ADP-ribose.

Docking of small molecule inhibitors. Docking experiments on the small molecule inhibitors A3,³⁰ M15,³⁰ (R)-sirtinol 9, (S)-sirtinol 10, and splitomicin³¹ were performed by the means of the Autodock program. To ascertain whether the above compounds could act as competitive or non-competitive inhibitors, docking was conducted using either the free Sir2-Af2 enzyme or the complex with one of the substrates. Although it has been recently reported that the NAD⁺ would likely bind only to the pre-formed Sir2/acetylated peptide binary complex,¹⁷ docking experiments were also undertaken using the less likely Sir2-Af2/NAD⁺ complex to ascertain the possible competitive inhibitory action against the Ac—K site.

Docking of A3. Against the free Sir2-Af2 enzyme and the Sir2-Af2/p53 complex, although in a reverse orientation, A3 occupies preferably the NAD⁺ binding site acting as a competitive inhibitor for this substrate. On the other hand, in the presence of NAD⁺, A3 seems to act primarily as non-competitive inhibitor, occupying a separate site, close but distinct from those of NAD⁺ and Ac—K binding sites. In the NAD⁺ competitive bound conformation the docked A3 molecule fully superimposes the NAD⁺ scaffold making favourable interaction with both hydrophobic and hydrophilic residues (Ala24, Gly25, Phe35, Arg36, Gln100, Asn101, His118, Gly191, Ser193). Thus it seems that A3 could act as non-competitive or NAD⁺ competitive inhibitor without affecting the acetylated peptide binding. In the non-competitive bound conformation the molecule of A3 seats in the nicotinamide escape tunnel³⁵ making favourable contacts with the side chains and backbone atoms of hydrophilic residues (Ser27, Lys73, Asn101, and Asp103).

Docking of M15. In the free Sir2-Af2 enzyme and the Sir2-Af2/NAD⁺ complex, Autodock finds the same binding conformation that seems to act as a competitive inhibitor for the Ac—K site. Differently, against the Sir2-Af2/p53 complex the docked M15 molecule only partially superimpose the NAD⁺ binding site. In the p53 competitive bound conformations the nitromethoxyphenyl moiety of M15 partially occupy the Ac—K binding site making favourable interactions with hydrophobic residues (Val162, Val163, Leu164 and Phe165), while the naphthol group interacts with the residue of the nicotinamide escape tunnel³⁵ making favourable contacts with the side chains and backbone atoms of Lys73, Asn101, Ile102 and Asp103. In the NAD⁺ competitive bound conformation the docked M15 only partially superimpose to the NAD⁺ binding site, suggesting that this inhibitor would likely not bind in the presence of the p53 substrate. Thus it seems that M15 could act as anti-Sir2 agent by inhibiting the binding of the acetylated substrate.

Docking of (R)-sirtinol (9). As seen for the previous docking of M15, also for (R)-sirtinol (9) Autodock suggest a similar binding conformation either in the free Sir2-Af2 enzyme or the Sir2-Af2/NAD⁺ complex. The molecule seems to act as a competitive inhibitor for the Ac—K site, but differently from what observed for M15. (R)-sirtinol (9) occupies the Ac—K entrance channel in a particular fashion overlapping part of the experimental acetylated p53 substrate. The two bound conformations differ only in the position of the 2-naphthol moiety, in particular the free enzyme bound conformation makes a π-stacking interaction with the Tyr197 while, in the 9 Sir2-Af2/NAD⁺ Autodock proposed binding mode, the 2-naphthol group is flipped about 180°, so that it lacks the π-stacking interaction but displays a strong hydrogen bond interaction with the carbonyl group of Tyr197 (distance OHₙₐₚₕₜₕₒₗ—O═C_{Tyr197}=1.911 Å). The other side of the two 9 conformations is perfectly superimposed displaying two further hydrogen bonds, a strong one between the 9 carboxamide oxygen and the Gly166 NH (distance C═O₂—HN_{Gly166}=2.032 Å) and a weak one between the carboxamide NH and the Tyr197 CO (distance N—H₂—O═C_{Tyr197}=2.407 Å). Furthermore the superimposition of the two (R)-sirtinol (9) bound conformations with the experimental acetylated p53 shows how the inhibitor can mime the substrate. In particular, the naphthol moiety partially occupies the spatial region of the p53 Leu12-Phe14; the central ortho-disubstituted benzene ring overlaps the hydrophobic parts of the Ac—K side chain; the carbonyl group plays the role of the Ac—K CO; the terminal phenyl ring overlaps the p53-Lys10.

The docking of 9 against the Sir2-Af2/p53 binary complex obviously places the molecule in a different space that superimpose partially the NAD⁺ without making visible important interaction with the complex, and suggesting that this NAD⁺ competitive inhibition mechanism is very unlikely.

Docking of (S)-sirtinol (10). Similar to that observed for the (R) enantiomer, Autodock suggests the (S)-sirtinol (10) to be a small molecule Sir2 inhibitor that competes for the Ac—K binding site. Again, similar to 9, the docked conformations obtained with the free and NAD⁺ bound enzyme are almost coincident, but they do not display the naphthol flipping. Upon superimposition of the two enantiomers, the naphthol of 10 is not co-planar with that of 9, and this does not allow to the (S) enantiomer to form either the OHₙₐₚₕₜₕₒₗ—O═C_{Tyr197} hydrogen bond or the π-stacking interaction. Moreover, the inversion of the chiral center makes the 10 carboxamide to be shifted not allowing the formation of the C═O_{carboxamide}—HN_{Gly166} hydrogen bond. In general, the two enantiomers 9 and 10 share a common binding mode, and thus they should show similar anti-Sir2 activities, nevertheless the difference in the details could explain a slightly higher activity for the (R)-sirtinol (9) over its specular form.

The docking of 10 against the Sir2-Af2/p53 binary complex, similarly as that observed for 9, shows 10 partially occupying the NAD⁺ binding site, thus suggesting that this binding mode is the only one possible in the presence of bound acetylated substrate.

Docking of splitomicin. By comparing the docking of splitomicin against the free Sir2-Af2 or the NAD⁺ and p53 binary complexes, one thing arises: splitomicin likely does not compete for the Ac—K binding site but rather acts to inhibit NAD⁺ binding. In docking experiments conducted against either the free enzyme or the p53 bound form splitomicin superimposes to the NAD⁺ structure placing the lacton moiety in proximity of the phosphodiester bridge, while the naphthalene ring overlaps the nicotinamide ribose (not shown). In the Sir2-Af2/NAD⁺ complex (the less likely)¹⁷ our modeling indicates that splitomicin binds in the nicotinamide escape channel³⁵ making contacts with surrounding residues Phe35, Gln100, Asn101, Ile102, Asp103 and His118.

Recently Hirao et al.⁴⁰ reported the manual docking of splitomicin (based on mutagenesis data) on a homology model of yeast Sir2p obtained from the human SIRT2 using the SwissModel server.⁴¹ We were not able in reproducing the homology model and thus the reported manual docking (the SwissModel server answered that there is too little identity between Sir2p and SIRT2 to obtain a reliable model). Nevertheless the residues conferring resistance to splitomicin (His286, Leu287 and Tyr298 of Sir2p)^{31,40} are part of a very flexible loop that could be of crucial importance in the open and closed enzyme form, influencing the loop flexibility and thus the approaching of splitomicin to the real binding site. In our docking experiment we modelled the flexible loop from the Sir2-Af2 opened form and therefore the molecule was allowed to approach the enzyme active site.

Sir2 Inhibitory Assay. The two sirtinol enantiomers 9 and 10 together with sirtinol (1) itself have been evaluated for their ability to inhibit yeast Sir2 (ySir2) enzyme. The results, expressed as the percent of inhibition at 100 μM and IC₅₀ (50% inhibitory concentration) values, are reported in Table 2 (above in Example 3).

When tested against yeast Sir2, sirtinol (1) showed 65.6% of inhibition at 100 μM, its IC₅₀ being 48 μM (Table 2 in Example 3). As expected from the analysis of their binding mode, (R)-sirtinol (9) and (S)-sirtinol (10) showed IC₅₀ values (9: 62 μM; 10: 66 μM) strictly related to 1 and each other, 9 being slightly more efficient than 10 in inhibiting Sir2 enzyme.

### CONCLUSION

We describe herein the Sir2-Af2/NAD⁺/p53 complex based on molecular modeling and docking studies. The obtained structure is in good agreement with the proposed mechanisms of action recently reported, and moreover suggests that His118 is extremely important for both the NAD⁺ molecular recognition during the formation of the ternary complex and in acetyl transfer. Furthermore, docking of five synthetic inhibitors both in the absence of Sir2 substrates or in the presence of p53 or NAD⁺ indicated that the five inhibitors might inhibit the Sir2 enzyme by via mechanisms. A3 and splitomicin seem to bind preferably in the NAD⁺ site or in a non-competitive side pocket blocking the nicotinamide escape, whereas (R)-(9) and (S)-sirtinol (10), by mimicking the backbone of the p53, might act as Ac—K binding-site competitors by blocking the entrance channel of the acetylated lysine. Lastly, M15 seems to bind in the bottom of the Ac—K binding site likely interfering with the transfer of the acetyl group, thereby acting as non-competitive inhibitor. The docking of these small molecules against the Sir2-Af2/NAD⁺ complex agree with a recent report by Borra et al.¹⁷ that is very unlikely that the free enzyme and the Sir2-Af2/p53 complex are the only forms that can be inhibited. Moreover, our docking experiments revealed that the (R)-sirtinol (9) could exert a slightly higher inhibitory potency than the (S)-sirtinol (10). Upon synthesis and testing against yeast Sir2, the two sirtinol enantiomers 9 and 10 resulted equally active in inhibiting the enzyme (Table 2 in Example 3), with 9 showing very slight higher IC₅₀ value than 10 as predicted by the docking studies.

### Materials and Methods

Chemistry. Melting points were determined on a Büchi 530 melting point apparatus and are uncorrected. Infrared (IR) spectra (KBr) were recorded on a Perkin-Elmer Spectrum One instrument. ¹H NMR spectra were recorded at 200 MHz on a Bruker AC 200 spectrometer; chemical shifts are reported in δ (ppm) units relative to the internal reference tetramethylsilane (Me₄Si). All compounds were routinely checked by TLC and ¹H NMR. TLC was performed on aluminum-backed silica gel plates (Merck DC-Alufolien Kieselgel 60 F₂₅₄) with spots visualized by UV light. All solvents were reagent grade and, when necessary, were purified and dried by standards methods. Concentration of solutions after reactions and extractions involved the use of a rotary evaporator operating at a reduced pressure of ca. 20 Torr. Organic solutions were dried over anhydrous sodium sulfate. Analytical results are within ±0.40% of the theoretical values. All chemicals were purchased from Aldrich Chimica, Milan (Italy) or Lancaster Synthesis GmbH, Milan (Italy) and were of the highest purity.

Syntheses. Samples prepared for physical and biological studies were dried in high vacuum over P₂O₅ for 20 h at temperatures ranging from 25 to 110° C., depending on the sample melting point.

General procedure for the synthesis of the 2-amino-N-(1-phenylethyl)benzamides 24 and 25. Example: (R)-2-amino-N-(1-phenylethyl)benzamide (24). A mixture of (R)-2-nitro-N-(1-phenylethyl)benzamide 21³⁷ (1.7 g, 6.3 mmol) and Pd/C (0.1 g) in 30 mL of dry ethanol was collected with hydrogenator reactor at 4.8 atm of H₂. After 2 h at room temperature, the solvent was evaporated under reduced pressure and the residue was recrystallized from CH₂Cl₂/hexane to furnish 24 as a pure product. ¹H NMR (CDCl₃) δ 1.55-1.60 (d, 3H, CHCH₃), 5.25-5.29 (m, 1H, CHCH₃), 6.23 (bs, 1H, NH exchangeable with D₂O), 6.64-6.69 (m, 2H, benzamide H-3,5), 7.19-7.37 (m, 7H, benzamide H-4,6 and phenyl). Anal. (C₁₅H₁₆N₂O)C, H, N.

General procedure for the synthesis of sirtinol compounds 1, 9, & 10. Example: (S)-2-[(2-hydroxy-1-naphthalenylmethylene)amino]-N-(1-phenylethyl)benzamide (10).

A mixture of 2-hydroxy-1-naphtaldehyde (0.5 g, 2.1 mmol) and (S)-2-amino-N-(1-phenylethyl)benzamide 25 (0.4 g, 2.1 mmol) in 30 mL of absolute ethanol:benzene (2:1) mixture in the presence of a catalytic amount of glacial acetic acid was heated at reflux for 4 h. After cooling at room temperature, from the mixture reaction a yellow solid was formed, which was collected by filtration, washed with CHCl₃ and purified by crystallization. ¹H NMR (DMSO-d₆) δ 1.40-1.41 (d, 3H, CHCH₃), 5.13-5.17 (q, 1H, CHCH₃), 6.94-6.96 (m, 1H, naphthyl H-3), 7.20-7.39 (m, 6H, Ph and naphthyl H-6), 7.50-7.54 (m, 1H, naphthyl H-7), 7.69-7.76 (m, 3H, benzene H-3,5 and naphthyl H-8), 7.89-7.91 (m, 1H, naphthyl H-5), 7.99-8.01 (m, 2H, benzene H-2,6), 8.46-8.48 (m, 1H, naphthyl H-4), 8.83-8.85 (m, 1H, CH═N), 9.64 (bs, 1H, OH). Anal. (C₂₆H₂₂N₂O₂) C, H, N.

In Vitro Yeast Sir2 Enzyme Inhibition. Recombinant His-tagged yeast Sir2p was purified and assayed for deacetylase activity using the HDAC fluorescent activity assay/drug discovery kit (AK-500, BIOMOL Research Laboratories).⁴² This assay system allows detection of a fluorescent signal upon deacetylation of a histone substrate when treated with developer. Fluorescence was measured on a fluorometric reader (Wallac Victor III fluorescence plate reader Perkin Elmer) with excitation set at 360 nm and emission detection set at 450 nm. Reactions consisted of 3 μg of ySir2 incubated with 250 μM acetylated histone substrate, 1 mM dithiothreitol, and a range of inhibitor concentrations as described. Reactions with the yeast and human proteins were carried out at 30 and 37° C., respectively, for 60 min. Assays were performed in the presence of 200 μM NAD⁺ and each of the inhibitors at 0, 20, 75, 100, 150, or 300 μM concentrations.

Molecular Modeling and Docking Studies. All molecular modeling calculations and manipulations were performed using the software packages Macromodel 7.1,⁴³ MOPAC 2000,^{44,45} Deep View Swiss-PdbViewer 3.7,⁴⁶ ADT 1.3, and Autodock 3.0.5^{48,49} running on IBM compatible AMD Athlon workstations. For the Image generation the program Chimera 1.2056⁵⁰ was used. For the conformational analysis and for any minimization, the all-atom Amber force field⁵¹ was adopted as implemented in the Macromodel package.

Sir2-Af2/NAD⁺/p53 Complex Structure Preparation. The Sir2-Af2/NAD⁺/p53 ternary complex was prepared starting from the Sir2-Af2/p53 complex reported by Avalos et al. (PDB entry code 1ma3).²⁷ However, the structure of the Sir2 homolog lacked of the 30-39 residues of the very flexible loop (aa 29-48) of the helical region.¹⁶ To fix the loop missing residues the structure of Sir2-Af1 reported by Chang et al. (PDB entry code 1m2g)¹⁶ was used. Using the Swiss-PdbViewer program the Sir2-Af2/p53 complex (1ma3) and the Sir2-Af1 were superimposed and the 30-39 decapeptide was copied into the 1ma3 structure. A molecular dynamic protocol was applied to equilibrate the obtained fixed Sir2-Af2/p53 complex as following: the structure was energy minimized to a low gradient. The non-bonded cut-off distances were set to 20 Å for both Van der Waals and electrostatic interactions. An initial random velocity to all atoms corresponding to 300 K was applied. Three subsequent molecular dynamics runs were then performed. The first was carried out for 50 ps with a 1.5 fs time step at a constant temperature of 300 K for equilibration purposes. Next, molecular dynamics was carried out for 50 ps, during which the system is coupled to a 150 K thermal bath with a time constant of 5 ps. The time constant represents approximately the half-life for equilibration with the bath; consequently, the second molecular dynamics command caused the molecule to slowly cool to approximately 150 K. The third and last dynamic cooled the molecule to 50 K over 50 ps. A final energy minimization was then carried out for 1000 iterations using conjugate gradient. The minimizations and molecular dynamics were in all cases performed in simulated aqueous solution using the batchmin GBSA keyword. Because of the presence of a metal Zn ion in the HDAC1 catalytic core and the intrinsic molecular mechanic electrostatic limitation of the AMBER force field, the whole molecular dynamic protocol was performed by applying AM1 charges calculated with the program MOPAC 2000 on the whole Sir2-Af2/p53 complex. Comparison of the 1ma3 and the 1m2g loop with the results of the SA dynamic runs afforded to very low RMSD values on the Ca traces, also including the residue side-chains, thus revealing that refined binary complex is a reliable choice for the applied docking studies. Finally to prepare the ternary complex the NAD⁺ molecule was docked by the mean of the Autodock program in the previously prepared binary complex. The initial NAD⁺ structure was extracted from the Sir2-Af1/NAD⁺ complex filed in the Brookheaven database (1ici)²⁶ and geometry optimized with the Macromodel program to a low gradient to obtain the conformation used in the docking run. The AutoDockTools package was employed to generate the docking input files and analyze the docking results; the same procedures as those described in the manual were followed. All the non-polar hydrogens and the water molecules were removed. The Kollmann charges were loaded for the proteins, while the all atom amber charges applied by the Macromodel program were retained in the ligand. A grid box size of 42×84×76 points with a spacing of 0.375 Å between the grid points was implemented and covered more than 5 Å of the NAD⁺ binding site. The grid was centered on the mass center of the experimentally bound NAD⁺ in the 1ici coordinates, previously aligned with Swiss-PdbView to the 1ma3 structure. All the single bonds were treated as active torsional bonds. One hundred docked structures, ie 100 runs, were generated by using genetic algorithm searches. A default protocol was applied, with an initial population of 50 randomly placed individuals, a maximum number of 2.5×10⁵ energy evaluations, and a maximum number of 2.7×10⁴ generations. A mutation rate of 0.02 and a crossover rate of 0.8 were used. Results differing by less than 2.0 Å in positional root-mean-square deviation (RMSD) were clustered together and represented by the result with the most favourable free energy of binding.

Because of Autodock is not able to perform any energy minimization of the generated complexes, the selection of the NAD⁺ binding conformation was not straightforward by using the first Autodock scored conformation. The selection of the binding conformation was performed by an energy based rescoring of the Autodock cluster represent conformations. The Macromodel program was used to minimize the corresponding 62 Autodock Sir2-Af2/NAD⁺/p53 complexes. The ligand and a 10 Å core of atoms around the NAD⁺ pocket were let to relax during the minimization. An external fixed shell of 8 Å was also included for the long-range interactions. The minimizations were performed applying AM1 charges calculated with the program MOPAC 2000. The global minimum from the above minimization was thus selected as the most likely Sir2-Af2/NAD⁺/p53 complex. Interestingly the NAD⁺ binding mode was comparable with the 1ici experimental bound conformation, and moreover was also comparable with the productive experimental NAD⁺ bound conformation in complex with the Sir2-Af2 recently reported by Avalos et al.³⁵ (see Result and Discussion section).

Small molecule inhibitors: structure preparation and docking. The structures of A3,³⁰ M15,³⁰ (R)-sirtinol (9), (S)-sirtinol (10), and splitomicin³¹ were drawn by the Macromodel graphical interface Maestro 3.0 and geometry optimized using a molecular dynamic protocol similar to that used in the optimization of the Sir2-Af2/p53 complex (see above). Each molecule was energy-minimized to a low gradient. The non-bonded cut-off distances were set to 20 Å for both van der Waals and electrostatic interactions. An initial random velocity to all atoms corresponding to 300 K was applied. Three subsequent molecular dynamics runs were then performed. The first was carried out for 10 ps with a 1.5 fs time step at a constant temperature of 300 K for equilibration purposes. The next molecular dynamic was carried out for 20 ps, during which the system is coupled to a 150 K thermal bath with a time constant of 5 ps. The time constant represents approximately the half-life for equilibration with the bath. Consequently the second molecular dynamic command caused the molecule to slowly cool to approximately 150 K. The third and last dynamic cooled the molecule to 50 K over 20 ps. A final energy minimization was then carried out for 250 iterations using a conjugate gradient. The minimizations and the molecular dynamics were in all cases performed using the continuum solvent simulation (GBSA keyword). The binding modes of the Sir2 small molecule inhibitors were analyzed by a docking procedure using the program Autodock. For the docking, a grid spacing of 0.375 Å and 70×72×72 number of points were used. The grid was centered on the mass center of the experimental p53 acetyl-lysine coordinates, and comprised more than 5 Å of both the NAD⁺ and Ac—K binding sites. The GA-LS method was adopted using the default settings. Amber united atoms for the protein and Gasteiger atom charge were for the inhibitors were assigned by the means of the program ADT. Autodock generated 100 possible binding conformations clustered in 2.0 Å. Three docking experiments were carried out for each inhibitor using the Sir2-Af2 enzyme in different compositions: (i) the free sir2-Af2 enzyme, (ii) the Sir2-Af2/NAD⁺ and (iii) Sir2-A 2/p53 binary complexes.

In parallel experiments either a capped Ac—K residue or p53 found in the PDB 1ma3 complex were docked back to the Sir-Af2 for docking assessment.

### REFERENCES FOR EXAMPLE 10

(1) Kurdistani et al. Nat. Rev. Mol. Cell Biol. 2003, 4, 276-284. (2) Wu et al. Trends Biochem. Sci. 2000, 25, 619-623. (3) Urnov et al. Emerg. Ther. Targets 2000, 4, 665-685. (4) Pazin et al. Cell 1997, 89, 325-328. (5) Grunstein et al. Nature 1997, 389, 349-352. (6) Davie et al. Curr. Opin. Genet. Dev. 1998, 8, 173-178. (7) Kouzarides et al. Curr. Opin. Genet. Dev. 1999, 9, 40-48. (8) Kouzarides et al. EMBO J. 2000, 19, 1176-1179. (9) Grozinger et al. Chem. Biol. 2002, 9, 3-16. (10) Mai et al. Med. Res. Rev. 2005, in press. (11) Bertos et al. Biochem. Cell Biol. 2001, 79, 243-252. (12) Fischle et al. Biochem. Cell Biol. 2001, 79, 337-348. (13) Verdin et al. Trends Genet. 2003, 19, 286-293. (14) Tanny et al. Cell 1999, 99, 735-745. (15) Tanner et al. Proc. Natl. Acad. Sci. USA 2000, 97, 14178-14182. (16) Chang et al. J. Biol. Chem. 2002, 277, 34489-34498. (17) Borra et al. Biochemistry 2004, 43, 9877-9887. (18) North et al. Genome Biol. 2004, 5, 224.1-224.10. (19) Blande et al. Annu. Rev. Biochem. 2004, 73, 417-435. (20) Gartemberg et al. Curr. Opin. Microbiol. 2000, 3, 132-137. (21) Guarente et al. Genes Dev. 2000, 14, 1021-1026. (22) Lin et al. Science 2000, 289, 2126-2128. (23) Tissenbaum et al. Nature 2001, 410, 227-230. (24) Brachmann et al. Gene Dev. 1995, 9, 2888-2902. (25) Mills et al. Cell 1999, 97, 609-620. (26) Min et al. Cell 2001, 105, 269-279. (27) Avalos et al. Mol. Cell 2002, 10, 523-535. (28) Finnin et al. Nat. Struct. Biol. 2001, 8, 621-625. (29) Zhao et al. Nat. Struct. Biol. 2003, 10, 864-871. (30) Grozinger et al. J. Biol. Chem. 2001, 276, 38837-38843. (31) Bedalov et al. Proc. Natl. Acad. Sci. USA 2001, 98, 15113-15118. (32) Sauve et al. Biochemistry 2001, 40, 15456-15463. (33) Landry et al. Proc. Natl. Acad. Sci. USA 2000, 97, 5807-5811. (34) Berman et al. Nucl. Acids Res. 2000, 28, 235-242. (35) Avalos et al. Mol. Cell 2004, 13, 639-648. (36) Bittennan et al. J. Biol. Chem. 2002, 47, 45099-45107. (37) Malyshev et al. J. ChromatogrA 1999, 859, 143-151. (38) Broggini et al. Tetrahedron-Asymmetry 1999, 10, 2203-2212. (39) Zhao et al. Proc. Natl. Acad. Sci. USA 2004, 101, 8563-8568. (40) Hirao et al. J. Biol. Chem. 2003, 278, 52773-52782. (41) Schwede et al. Nucl. Acids Res. 2003, 31, 3381-3385. (42) Howitz et al. Nature 2003, 425, 191-196. (43) Mohamad et al. J. Comput. Chem. 1990, 11, 440-467. (44) Stewart et al. J. Comput.-Aided Mol. Des. 1990, 4, 1-105. (45) MOPAC 2000.00 Manual; Stewart, J. J. P., Ed; Fujitsu Limited: Tokyo, Japan 1999. (46) Guex et al. Electrophoresis 1997, 18, 2714-2723. (see the expasy website on the world wide web with the extension expasy.org/spdbv.). (47) Sanner et al. Proc. Pacific Symposium Biocomputing 1998, pp 401-412. (48) Goodsell et al. J. Mol Recognit. 1996, 9, 1-5. (49) Oshiro et al. J. Comput.-Aided Mol. Des. 1995, 9, 113-130. (50) Pettersen J. Comput. Chem. 2004, 25, 1605-1612. (also see the UCSF Chimera website on the world wide web with the extension cgl.ucsf.edu/chimera.). (51) Pearlman Comput. Phys. Commun. 1995, 91, 1-41.

### Example 11

### Inhibition of SIRT2

This Example shows that compounds (1), (9), (10), (8) and (7) also inhibit the human sirtuin SIRT2.

Recombinant His-tagged SIRT2 isoform 1 was purified and assayed for deacetylase activity using the HDAC fluorescent activity assay/drug discovery kit (AK-500, BIOMOL Research Laboratories) as described above. 100 μM of the compounds (1), (9), (10), (8) and (7) were added in the reaction. A dose response curve for determining the IC₅₀ was also done.

The results are shown below in Table 4:

TABLE 4
SIRT2 inhibition by compounds 1, 9, 10, 8 and 7

| Compound | SIRT2 % inhibition at 100 μM | IC50 (μM) |
|---|---|---|
| 1 (Sirtinol) | 71.0 +/− 8 | 57.7 +/− 9 |
| 9 (R-Sirtinol) | 72.2 +/− 4 | 49.3 +/− 6 |
| 10 (S-Sirtinol) | 73.5 +/− 4 | 39.4 +/− 5 |
| 8 (para-Sirtinol) | 80.2 +/− 7 | 25.9 +/− 6 |
| 7 (meta-Sirtinol) | 79.2 +/− 10 | 35.7 +/− 2 |

Similarly to the data obtained with SIRT1, para-Sirtinol appears to be the most potent on SIRT2.

1 is a better inhibitor of human SIRT2 than hSIRT1. Both m-sirtinol (7) and p-sirtinol (8) were more potent than 1 in inhibiting SIRT2 (from 1.6 fold (7) to 2.2 fold (8)), while 9 and 10 showed similar IC50 values, 10 being slightly more active (1.5 fold) than 1.

### CONCLUSION

A series of sirtinol analogues 2-10 have been synthesized, and their ability to inhibit yeast Sir2, human SIRT1 and human SIRT2 has been compared to that of sirtinol (1). Phenotypic assay, based on the inhibition of Sir2-mediated telomeric URA3 gene silencing was performed on 1 and 7-10. In vitro assays showed that compounds 2-4 and 6, lacking the 2-hydroxy group at the naphthalene moiety (2) or bearing several modifications at the benzene 2′-position of the aniline portion (carbethoxy (3), carboxy (4), and cyano (6)) of sirtinol 1, were 3- to 13-fold less potent than 1 against yeast Sir2 or were 1.3- to 5-fold less potent against human SIRT1. The 2′-carboxamido analogue 5 was totally inactive in both enzyme assays. 3-[2-Hydroxy-1-naphthalenyl-methylene]amino]-N-(1-phenylethyl)benzamide 7 and 4-[(2-hydroxy-1-naphthalenylmethylene)amino]-N-(1-phenylethyl)benzamide 8 (m- and p-sirtinol, respectively) have IC50 values similar to those of 1 for yeast Sir2, while against hSIRT1 and hSIRT2 they were from 1.6 to 10 times more potent than 1. Furthermore, in phenotypic screening 7 and 8 were endowed with the same potent Sir2 inhibitory as 1. Interestingly, in the 7 and 8 structures, the hydrogen bond observed in sirtinol 1 between the naphthyl 2-hydroxy group, the aldimine nitrogen, and the carbonyl of the amide function is considerably less favorable.

Sir2 and Sir2-like inhibitory assays as well as phenotypic screening showed that there is no enantioselective effect of (R)-sirtinol (9) and (S)-sirtinol (10), 9 and 10 inhibitory data being similar to each other as well as to 1. in the hSIRT1 assay, 9 and 10 were 2-fold more potent than 1 in inhibiting the enzyme.

### INCORPORATION BY REFERENCE

All publication, patents, and patent applications mentioned herein are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the world wide web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound of formula I: 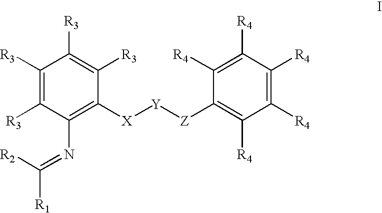
wherein, independently for each occurrence,
X is —O—, —N(Rₐ)—, —C(Rₐ)₂—, —C(═O)—, —C(═NR_{b})—, —C(═S)—, —S—, —S(═O)— or —S(═O)₂—;
Y is —O—, —N(Rₐ)—, —C(Rₐ)₂—, —C(═O)—, —C(═NR_{b})—, —C(═S)—, —S—, —S(═O)— or —S(═O)₂—;
Z is —O—, —N(Rₐ)—, —C(Rₐ)₂—, —C(═O)—, —C(═NR_{b})—, —C(═S)—, —S—, —S(═O)— or —S(═O)₂—;
Rₐ is hydrogen, alkyl, aryl, or aralkyl;
R_{b} is hydrogen, hydroxyl, alkoxyl, amine, alkyl, aryl, or aralkyl;
R₁ is aryl;
R₂ is hydrogen, alkyl, aryl, or aralkyl;
R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl, or sulfoxido;
R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl, or sulfoxido; provided that when X is —C(═O)—; Y is —N(H)—; Z is —CH(CH₃)—; R₂ is hydrogen; R₃ is hydrogen; and R₄ is hydrogen; R₁ is not 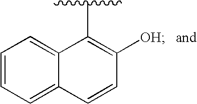
the compound is achiral or, when chiral, is a single stereoisomer or a mixture of stereoisomers;
or
a compound of formula II: 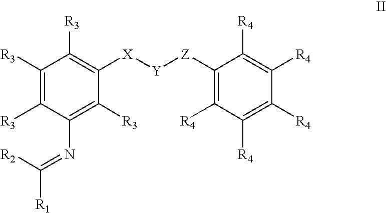
wherein, independently for each occurrence,
X is —O—, —N(Rₐ)—, —C(Rₐ)₂—, —C(═O)—, —C(═NR_{b})—, —C(═S)—, —S—, —S(═O)— or —S(═O)₂—;
Y is —O—, —N(Rₐ)—, —C(Rₐ)₂—, —C(═O)—, —C(═NR_{b})—, —C(═S)—, —S—, —S(═O)— or —S(═O)₂—;
Z is —O—, —N(Rₐ)—, —C(Rₐ)₂—, —C(═O)—, —C(═NR_{b})—, —C(═S)—, —S—, —S(═O)— or —S(═O)₂—;
Rₐ is hydrogen, alkyl, aryl, or aralkyl;
R_{b} is hydrogen, hydroxyl, alkoxyl, amine, alkyl, aryl, or aralkyl;
R₁ is aryl;
R₂ is hydrogen, alkyl, aryl, or aralkyl;
R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido; and
the compound is achiral or, when chiral, is a single stereoisomer or a mixture of stereoisomers;
or
a compound of formula III: 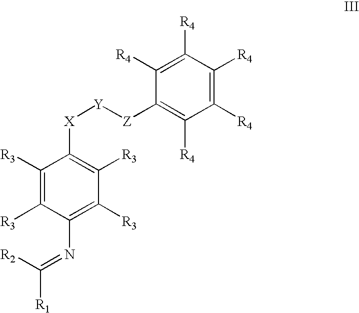
wherein, independently for each occurrence,
X is —O—, —N(Rₐ)—, —C(Rₐ)₂—, —C(═O)—, —C(═NR_{b})—, —C(═S)—, —S—, —S(═O)— or —S(═O)₂—;
Y is —O—, —N(Rₐ)—, —C(Rₐ)₂—, —C(═O)—, —C(═NR_{b})—, —C(═S)—, —S—, —S(═O)— or —S(═O)₂—;
Z is —O—, —N(Rₐ)—, —C(Rₐ)₂—, —C(═O)—, —C(═NR_{b})—, —C(═S)—, —S—, —S(═O)— or —S(═O)₂—;
Rₐ is hydrogen, alkyl, aryl, or aralkyl;
R_{b} is hydrogen, hydroxyl, alkoxyl, amine, alkyl, aryl, or aralkyl;
R₁ is aryl;
R₂ is hydrogen, alkyl, aryl, or aralkyl;
R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido; and
the compound is achiral or, when chiral, is a single stereoisomer or a mixture of stereoisomers,
or
a compound of formula IV: 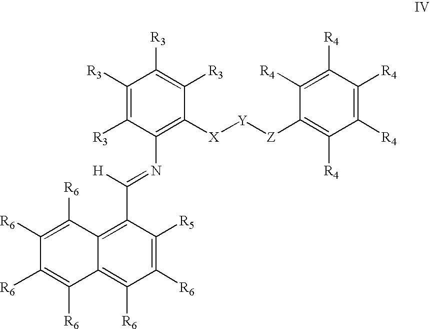
wherein, independently for each occurrence,
X is —O—, —N(Rₐ)—, —C(Rₐ)₂—, —C(═O)—, —C(═NR_{b})—, —C(═S)—, —S—, —S(═O)— or —S(═O)₂—;
Y is —O—, —N(Rₐ)—, —C(Rₐ)₂—, —C(═O)—, —C(═NR_{b})—, —C(═S)—, —S—, —S(═O)— or —S(═O)₂—;
Z is —O—, —N(Rₐ)—, —C(Rₐ)₂—, —C(═O)—, —C(═NR_{b})—, —C(═S)—, —S—, —S(═O)— or —S(═O)₂;
Rₐ is hydrogen, alkyl, aryl, or aralkyl;
R_{b} is hydrogen, hydroxyl, alkoxyamine, alkyl, aryl, or aralkyl;
R₁ is aryl;
R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₅ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₆ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
provided that when X is —C(═O)—; Y is —N(H)—; Z is —CH(CH₃)—; R₃ is hydrogen; R₄ is hydrogen; and R₆ is hydrogen; R₅ is not hydroxyl; and
the compound is achiral or, when chiral, is a single stereoisomer or a mixture of stereoisomers;
or
a compound of formula V: 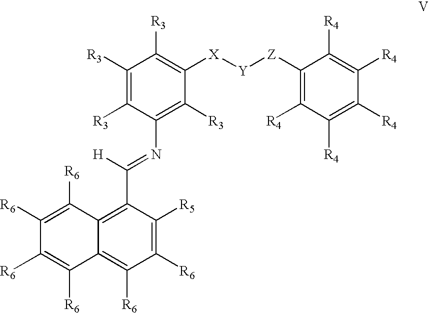
wherein, independently for each occurrence,
X is —O—, —N(Rₐ)—, —C(Rₐ)₂—, —C(═O)—, —C(═NR_{b})—, —C(═S)—, —S—, —S(═O)— or —S(═O)₂—;
Y is —O—, —N(Rₐ)—, —C(Rₐ)₂—, —C(═O)—, —C(═NR_{b})—, —C(═S)—, —S—, —S(═O)— or —S(═O)₂—;
Z is —O—, —N(Rₐ)—, C(Rₐ)₂—, —C(═O)—, —C(═NR_{b})—, —C(═S)—, —S—, —S(═O)— or —S(═O)₂—;
Rₐ is hydrogen, alkyl, aryl, or aralkyl;
R_{b} is hydrogen, hydroxyl, alkoxyamine, alkyl, aryl, or aralkyl;
R₁ is aryl;
R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₅ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₆ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido; and
the compound is achiral or, when chiral, is a single stereoisomer or a mixture of stereoisomers;
or
a compound of formula VI: 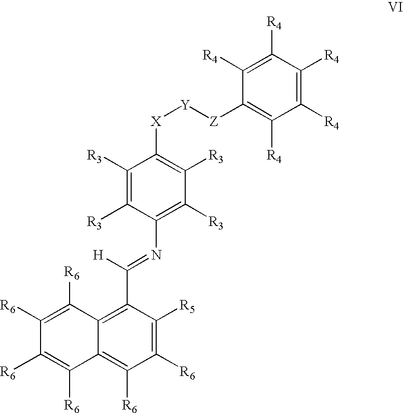
wherein, independently for each occurrence,
X is —O—, —N(Rₐ)—, —C(Rₐ)₂—, —C(═O)—, —C(═NR_{b})—, —C(═S)—, —S—, —S(═O)— or —S(═O)₂—;
Y is —O—, —N(Rₐ)—, —C(Rₐ)₂—, —C(═O)—, —C(═NR_{b})—, —C(═S)—, —S—, —S(═O)— or —S(═O)₂—;
Z is —O—, —N(Rₐ)—, —C(Rₐ)₂—, —C(═O)—, —C(═NR_{b})—, —C(═S)—, —S—, —S(═O)— or —S(═O)₂—;
Rₐ is hydrogen, alkyl, aryl, or aralkyl;
R_{b} is hydrogen, hydroxyl, alkoxyamine, alkyl, aryl, or aralkyl;
R₁ is aryl;
R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₅ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₆ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido; and
the compound is achiral or, when chiral, is a single stereoisomer or a mixture of stereoisomers;
or
a compound of formula VII: 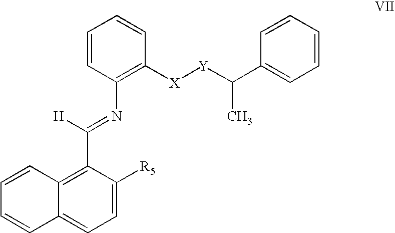
wherein, independently for each occurrence,
X is —N(H)—, —C(═O)—, —S— or —S(═O)₂—;
Y is —N(H)—, —CH₂— or —C(═O)—;
R₅ is hydrogen, hydroxyl or alkoxyl;
provided that when X is —C(═O)—; and Y is —N(H)—; R₅ is not hydroxyl; and
the compound is achiral or, when chiral, is a single stereoisomer or a mixture of stereoisomers;
or
a compound of formula VIII: 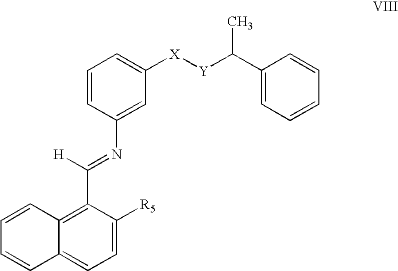
wherein, independently for each occurrence,
X is —N(H)—, —C(═O)—, —S— or —S(═O)₂—;
Y is —N(H)—, —CH₂— or —C(═O)—;
R₅ is hydrogen, hydroxyl or alkoxyl; and
the compound is achiral or, when chiral, is a single stereoisomer or a mixture of stereoisomers;
or
a compound of formula IX: 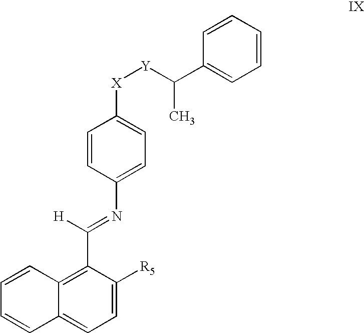
wherein, independently for each occurrence,
X is —N(H)—, —C(═O)—, —S— or —S(═O)₂—;
Y is —N(H)—, —CH₂— or —C(═O)₂—;
R₅ is hydrogen, hydroxyl or alkoxyl; and
the compound is achiral or, when chiral, is a single stereoisomer or a mixture of stereoisomers;
or
a compound of formula X: 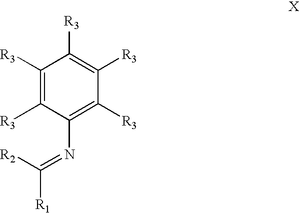
wherein, independently for each occurrence,
R₁ is aryl;
R₂ is hydrogen, alkyl, aryl, or aralkyl;
R₃ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
R₄ is hydrogen, halogen, alkyl, alkenyl, alkynyl, aryl, heteroaryl, aralkyl, aralkyenyl, aralkynyl, heteroaralkyl, heteroaralkyenyl, heteroaralkynyl, cyano, nitro, sulfhydryl, hydroxyl, sulfonyl, amino, acylamino, amido, alkylthio, carboxyl, carbamoyl, alkoxyl, sulfonate, sulfate, sulfonamido, sulfamoyl, sulfonyl or sulfoxido;
provided that when R₂ is hydrogen; R₃ is hydrogen; and R₄ is —C(═O)NHCH(CH₃)Ph;
R₁ is not 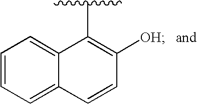
the compound is achiral or, when chiral, is a single stereoisomer or a mixture of stereoisomers;
or
a compound of formula XI: 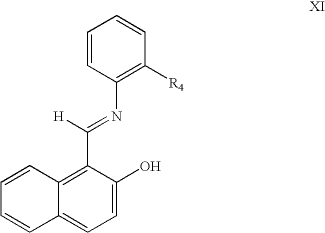
wherein, independently for each occurrence,
R₄ is —C(═O)ORₐ, —C(═O)N(Rₐ)₂ or —CN;
Rₐ is hydrogen alkyl, aryl, or aralkyl;
provided that R₄ is not —C(═O)NHCH(CH₃)Ph; and
the compound is achiral or, when chiral, is a single stereoisomer or a mixture of stereoisomers.

2. The compound of claim 1, having formula I, II or III, wherein X is —C(═O)—, —N(H)—, —S— or —S(═O)₂—.

3. The compound of claim 1, having formula I, II or III, wherein Y is —C(═O)—, —N(H)— or —CH₂—.

4. The compound of claim 1, having formula I, II or III, wherein Z is —CH(CH₃)—.

5. The compound of claim 1, having formula I, II or III, wherein R₂ is hydrogen.

6. The compound of claim 1, having formula I, II or III, wherein R₃ is hydrogen.

7. The compound of claim 1, having formula I, II or III, wherein R₄ is hydrogen.

8. The compound of claim 1, having formula I, II or III, wherein R₂ is hydrogen; R₃ is hydrogen; and R₄ is hydrogen.

9. (canceled)

10. The compound of claim 1, having formula IV, V or VI, wherein X is —C(═O)—, —N(Rₐ)—, —S— or —S(═O)₂—.

11. The compound of claim 1, having formula IV, V or VI, wherein X is —C(═O)—, —N(H)—, —S— or —S(═O)₂—.

12. The compound of claim 1, having formula IV, V or VI, wherein Y is —C(═O)—, —N(Rₐ)— or —C(Rₐ)₂—.

13. The compound of claim 1, having formula IV, V or VI, wherein Y is —C(═O)—, —N(H)— or —CH₂—.

14. The compound of claim 1, having formula IV, V or VI, wherein Z is —C(Rₐ)₂—.

15. The compound of claim 1, having formula IV, V or VI, wherein Z is —CH(Rₐ)—; and Rₐ is alkyl.

16. The compound of claim 1, having formula IV, V or VI, wherein Z is —CH(CH₃)—.

17. The compound of claim 1, having formula IV, V or VI, wherein R₃ is hydrogen, R₄ is hydrogen, R₅ is hydroxyl and/or R₆ is hydrogen.

18. The compound of claim 1, having formula IV, V or VI, wherein R₅ is hydroxyl; and R₆ is hydrogen.

19. The compound of claim 1, having formula IV, V or VI, wherein R₅ is hydroxyl; R₆ is hydrogen; and R₄ is hydrogen.

20. The compound of claim 1, having formula IV, V or VI, wherein R₅ is hydroxyl; R₆ is hydrogen; R₄ is hydrogen; and R₃ is hydrogen.

21. The compound of claim 1, having formula IV, V or VI, wherein R₅ is hydroxyl; R₆ is hydrogen; R₄ is hydrogen; R₃ is hydrogen; Z is —CH(Rₐ)—; and Rₐ is alkyl.

22. The compound of claim 1, having formula IV, V or VI, wherein R₅ is hydroxyl; R₆ is hydrogen; R₄ is hydrogen; R₃ is hydrogen; and Z is —CH(CH₃)—.

23. (canceled)

24. The compound of claim 1, having formula VII, VII or IX, wherein R₅ is hydroxyl.

25. The compound of claim 1, having formula VII, VII or IX, wherein R₅ is hydroxyl; X is —C(═O)—; and Y is —N(H)—.

26. The compound of claim 1, having formula VII, VII or IX, wherein R₅ is hydroxyl; X is —N(H)—; and Y is —C(═O)—.

27. The compound of claim 1, having formula VII, VII or IX, wherein R₅ is hydroxyl; X is —S—; and Y is —CH₂—.

28. The compound of claim 1, having formula VII, VII or IX, wherein R₅ is hydroxyl; X is —S(═O)₂—; and Y is —N(H)—.

29. The compound of claim 1, having formula VII, VII or IX, wherein the compound is a single enantiomer or stereoisomer.

30. (canceled)

31. The compound of claim 1, having formula X or XI, wherein R₄ is —C(═O)OEt, —C(═O)OH, —C(═O)NH₂ or —CN.

32. A composition comprising a first agent that is a compound of claim 1 and a second agent.

33. The composition of claim 32, wherein the second agent is a compound having any one of formulas I-XI.

34. The composition of claim 33, wherein the second agent is a chemotherapeutic agent.

35. A method for inhibiting a sirtuin, comprising contacting the sirtuin with a compound of claim 1.

36. The method of claim 35, wherein the sirtuin is SIRT1 or SIRT2.

37. A method for inhibiting a sirtuin in a cell, comprising contacting a cell comprising a sirtuin with a compound of claim 1.

38. The method of claim 37, wherein the sirtuin is SIRT1 or SIRT2.

39. A method for reducing the lifespan of a cell, killing a cell, or rendering a cell more sensitive to stress, comprising contacting the cell with a compound of claim 1.

40. The method of claim 39, wherein the stress is exposure to an agent that induces cell death.

41-43. (canceled)

44. A method for inhibiting tubule deacetylation in a cell, comprising contacting the cell with a SIRT2 inhibitory compound having a formula selected from the group consisting of formulas I, III, IV, VI, VII or IX.


SEQUENCE LISTING (6 sequences)

SEQ ID NO: 1 - DNA, 4107 bp, Homo sapiens
  CDS (54)..(2297)
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaaaaaaaaaaaaaaaaaaaa

SEQ ID NO: 2 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 3 - DNA, 1963 bp, Homo sapiens
  CDS (201)..(1370)
gtgttgtacgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagacccctctcaccctctggagacccaggcagggaaggtgcaggaggctcaggactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaac

SEQ ID NO: 4 - protein, 389 aa, Homo sapiens
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 5 - DNA, 1931 bp, Homo sapiens
  CDS (257)..(1315)
cgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagaccgactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaacaaaaaaaaaaaaaaaaaaaaaaa

SEQ ID NO: 6 - protein, 352 aa, Homo sapiens
MDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ